(19) United States
(12) Patent Application Publication
(10) Pub. No.: US 2023/0120542 A1
(43) Pub. Date: Apr. 20, 2023

(54) PHOTO-DECOMPOSABLE COMPOUND, PHOTORESIST COMPOSITION INCLUDING THE SAME, AND METHOD OF MANUFACTURING INTEGRATED CIRCUIT DEVICE

(71) Applicant: SAMSUNG ELECTRONICS CO., LTD., Suwon-si (KR)

(72) Inventors: Sumin KIM, Suwon-si (KR); Hyunwoo KIM, Seongnam-si (KR); Sukkoo HONG, Suwon-si (KR); Yechan KIM, Hwaseong-si (KR); Juyoung KIM, Busan (KR); Jinjoo KIM, Seoul (KR); Juhyeon PARK, Suwon-si (KR); Hyunji SONG, Anyang-si (KR); Songse YI, Seoul (KR)

(21) Appl. No.: 18/080,348

(22) Filed: Dec. 13, 2022

Related U.S. Application Data
(63) Continuation of application No. 17/005,636, filed on Aug. 28, 2020.

(30) Foreign Application Priority Data
Jan. 29, 2020 (KR) 10-2020-0010484

(51) Int. Cl. G03F 7/004; C07C 381/12; G03F 7/039; C07C 309/12

(52) U.S. Cl. CPC G03F 7/0045; C07C 381/12; G03F 7/0392; C07C 309/12; C07C 2603/74; C07C 2601/08

(57) ABSTRACT

A photo-decomposable compound includes an anion component including an adamantyl group and a cation component including a C5 to C40 cyclic hydrocarbon group and forming a complex with the anion component. At least one of the adamantyl group and the cyclic hydrocarbon group has a substituent, which decomposes by acid and generates an alkali soluble group. The substituent includes an acid-labile protecting group. A photoresist composition includes a chemically amplified polymer, the photo-decomposable compound, and a solvent. To manufacture an integrated circuit (IC) device, a photoresist film is formed using the photoresist composition on a feature layer, a first area of the photoresist film is exposed to generate a plurality of acids from the photo-decomposable compound in the first area, the chemically amplified polymer is deprotected due to the plurality of acids, and the first area is removed to form a photoresist pattern.

### CROSS-REFERENCE TO RELATED APPLICATIONS

This is a continuation application based on pending application Ser. No. 17/005,636, filed Aug. 28, 2020, the entire contents of which is hereby incorporated by reference.

Korean Patent Application No. 10-2020-0010484, filed on Jan. 29, 2020, in the Korean Intellectual Property Office, and entitled: “Photo-Decomposable Compound, Photoresist Composition Including the Same, and Method of Manufacturing Integrated Circuit Device,” is incorporated by reference herein in its entirety.

### BACKGROUND

### 1. Field

Embodiments relate to a photo-decomposable compound, a photoresist composition including the same, and a method of manufacturing an integrated circuit (IC) device.

### 2. Description of the Related Art

As IC devices have rapidly been downscaled and highly integrated, techniques for ensuring the dimensional precision of a pattern to be formed when the pattern is formed using a photolithography process have been considered.

### SUMMARY

The embodiments may be realized by providing a photo-decomposable compound including an anion component including an adamantyl group; and a cation component including a C5 to C40 cyclic hydrocarbon group, the cation component forming a complex with the anion component, wherein at least one of the adamantyl group and the C5 to C40 cyclic hydrocarbon group has a substituent, the substituent decomposes in response to exposure to an acid to generate an alkali soluble group, and the substituent includes an acid-labile protecting group.

The embodiments may be realized by providing a photoresist composition including a chemically amplified polymer; a solvent; and a photo-decomposable compound that includes an anion component including an adamantyl group and a cation component including a C5 to C40 cyclic hydrocarbon group, the cation component forming a complex with the anion component, wherein at least one of the adamantyl group and the C5 to C40 cyclic hydrocarbon group has a substituent that decomposes by an action of acid and generates an alkali soluble group, and the substituent includes an acid-labile protecting group.

The embodiments may be realized by providing a method of manufacturing an integrated circuit (IC) device, the method including providing a substrate that includes a feature layer; forming a photoresist film on the feature layer, wherein the photoresist film includes a chemically amplified polymer, a photo-decomposable compound, and a solvent, the photo-decomposable compound has an anion component including an adamantyl group and a cation component including a C5 to C40 cyclic hydrocarbon group and forming a complex with the anion component, at least one of the adamantyl group and the C5 to C40 cyclic hydrocarbon group has a substituent, the substituent decomposes in response to an acid to generate an alkali soluble group, and the substituent includes an acid-labile protecting group; exposing a first area of the photoresist film to generate a plurality of acids from the photo-decomposable compound in the first area, and deprotecting the chemically amplified polymer due to the plurality of acids, the first area being a portion of the photoresist film; removing the exposed first area of the photoresist film using a developer to form a photoresist pattern, the photoresist pattern including a non-exposed area of the photoresist film; and processing the feature layer using the photoresist pattern.

### BRIEF DESCRIPTION OF THE DRAWINGS

Features will be apparent to those of skill in the art by describing in detail exemplary embodiments with reference to the attached drawings in which:

FIG. 1 is a flowchart of a method of manufacturing an integrated circuit (IC) device, according to embodiments; and

FIGS. 2A to 2F are cross-sectional views of stages in a method of manufacturing an IC device, according to embodiments.

### DETAILED DESCRIPTION

A photo-decomposable compound according to embodiments may include an anion component including an adamantyl group and a cation component, which includes a C5 to C40 cyclic hydrocarbon group and forms a complex with the anion component. At least one of the adamantyl group included in the anion component and the C5 to C40 cyclic hydrocarbon group included in the cation component may have a substituent that decomposes by action of acid (e.g., in response to being exposed to acid) and generates an alkali soluble group. The substituent may include an acid-labile protecting group.

In an implementation, the acid-labile protecting group included in the photo-decomposable compound according to the embodiments may be selected from a substituted or unsubstituted t-butyl group and a C3 to C30 substituted or unsubstituted tertiary alicyclic group.

As used herein, unless otherwise defined, the term “substituted” may refer to including at least one substituent, e.g., a halogen atom (e.g., a fluorine (F) atom, a chlorine (Cl) atom, a bromine (Br) atom, or iodine (I) atom), hydroxyl, amino, thiol, carboxyl, carboxylate, ester, amide, nitrile, sulfide, disulfide, nitro, C1 to C20 alkyl, C1 to C20 cycloalkyl, C2 to C20 alkenyl, C1 to C20 alkoxy, C2 to C20 alkenoxy, C6 to C30 aryl, C6 to C30 aryloxy, C7 to C30 alkylaryl, or a C7 to C30 alkylaryloxy group. As used herein, the term “or” is not an exclusive term, e.g., “A or B” would include A, B, or A and B.

In an implementation, the acid-labile protecting group included in the photo-decomposable compound according to the embodiments may have an unsubstituted structure. In an implementation, the acid-labile protecting group may include an unsubstituted t-butyl group or a C3 to C30 unsubstituted tertiary alicyclic group.

In an implementation, the acid-labile protecting group included in the photo-decomposable compound according to the embodiments may have a structure substituted with a first substituent. In an implementation, the acid-labile protecting group may include a t-butyl group substituted with the first substituent or a C3 to C30 tertiary alicyclic group substituted with the first substituent. The first substituent may include, e.g., a C1 to C10 alkyl group, a C1 to C10 alkoxy group, a halogen atom, a C1 to C10 halogenated alkyl group, a hydroxyl group, an unsubstituted C6 to C30 aryl group, or a C6 to C30 aryl group in which some of carbon atoms included in the first substituent is substituted with a halogen atom or a heteroatom-containing group. The halogen atom that may be included in the first substituent may be a F atom, a C1 atom, a Br atom, or an I atom. The halogenated alkyl group may include a halogen atom, e.g., the F atom, the C1 atom, the Br atom, or the I atom. The heteroatom may be an oxygen atom, a sulfur atom, or a nitrogen atom. In an implementation, the heteroatom-containing group may be, e.g., —O—, —C(═O)—O—, —O—C(═O)—, —C(═O)—, —O—C(═O)—O—, —C(═O)—NH—, —NH—, —S—, —S(═O)₂—, or —S(═O)₂—O—.

In an implementation, the substituent, which is included in the photo-decomposable compound according to the embodiments and decomposes by the action of acid to generate the alkali soluble group, may have one of the following structures.
*—C(═O)OR¹
*—OC(═O)OR¹
*—OAc

R¹ may be an acid-labile protecting group, e.g., a substituted or unsubstituted t-butyl group or a C3 to C30 substituted or unsubstituted tertiary alicyclic group, Ac may be an acetal protecting group, and “*” indicates a bonding site.

In an implementation, R¹ may be a tertiary alicyclic group including an alicyclic hydrocarbon group. In an implementation, R¹ may include a group in which two hydrogen atoms are excluded from C3 to C12 (e.g., C3 to C6 or C5 to C12) monocycloalkane. In an implementation, R¹ may be a tertiary monocycloalkane (e.g., including 5 to 12 ring carbon atoms). In an implementation, R¹ may be a tertiary alicyclic group including an alicyclic hydrocarbon group. The alicyclic hydrocarbon group may include a group in which two hydrogen atoms are excluded from C7 to C12 polycycloalkane. In an implementation, R¹ may be a tertiary polycycloalkane.

In an implementation, R¹ may be one of the following structures.

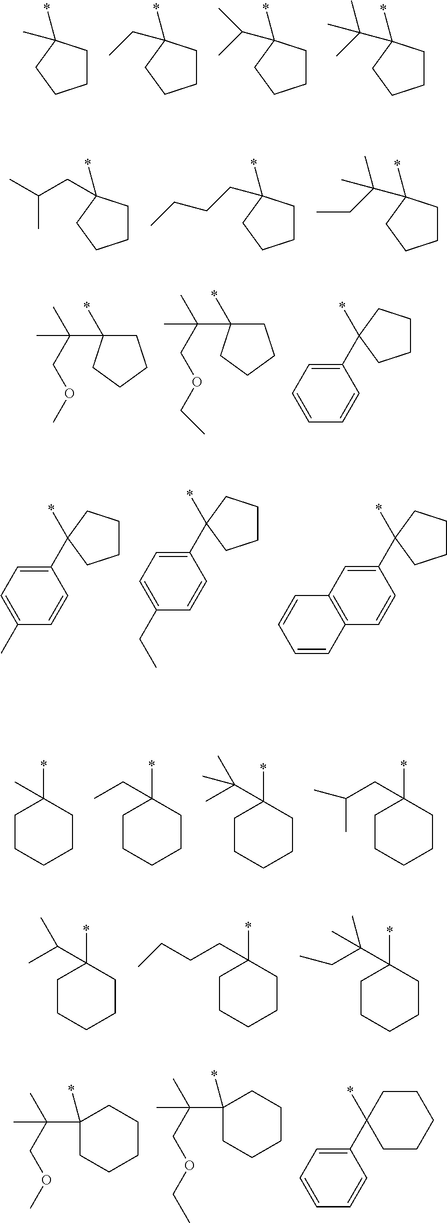

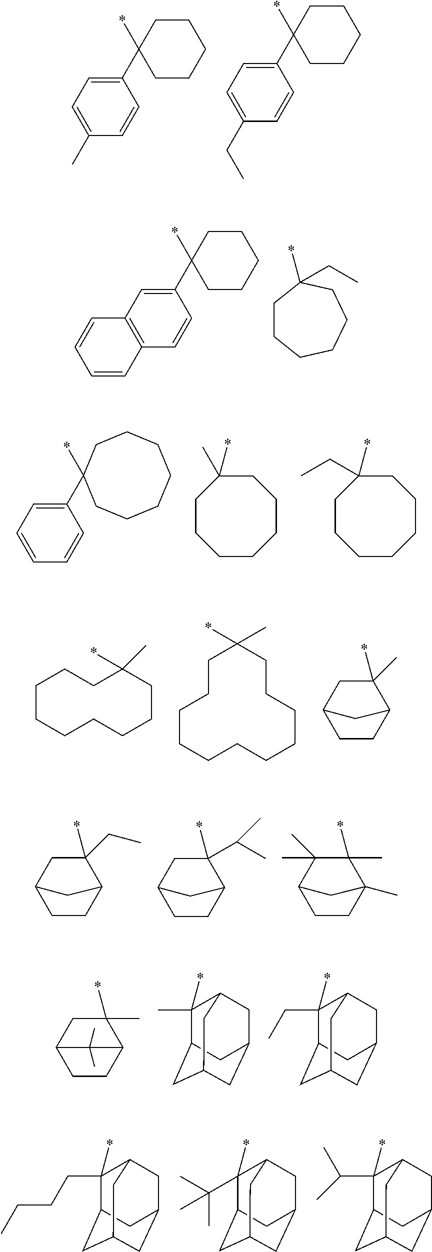

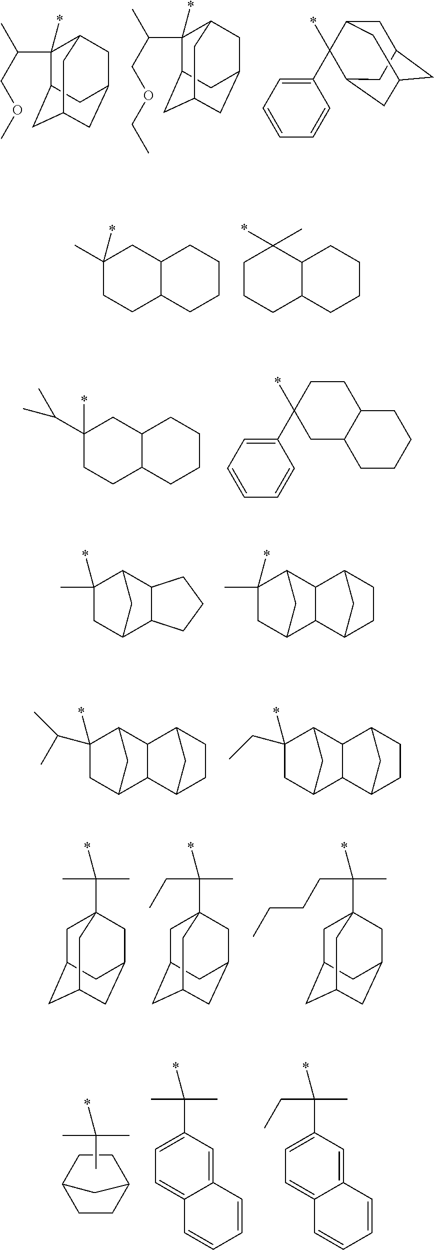

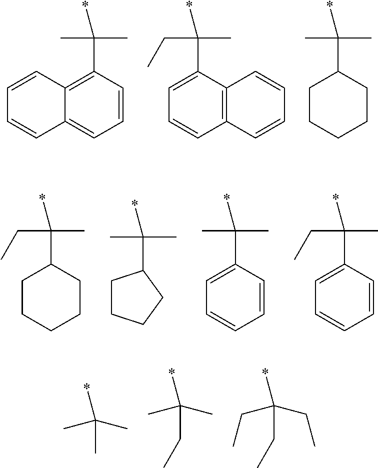

“*” indicates a bonding site.

In an implementation, the acetal protecting group may include a 1-alkoxyalkyl group. In an implementation, the acetal protecting group may include, e.g., a 1-ethoxyethyl group, a 1-methoxyethyl group, a 1-n-butoxyethyl group, a 1-isobutoxyethyl group, a 1-(2-chloroethoxy)ethyl group, a 1-(2-ethylhexyloxy)ethyl group, a 1-n-propoxyethyl group, a 1-cyclohexyloxyethyl group, a 1-(2-cyclohexylethoxy)ethyl group, or a 1-benzyloxyethyl group.

In an implementation, the substituent may be connected to the adamantyl group of the photo-decomposable compound. In an implementation, the photo-decomposable compound may be represented by Formula 1.

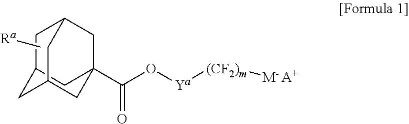

R^{a} may be a substituent, which decomposes by the action of acid and generates an alkali soluble group, and is represented by *—C(═O)OR¹. In an implementation, R¹ denotes an acid-labile protecting group, and may include a substituted or unsubstituted t-butyl or a C3 to C30 substituted or unsubstituted tertiary alicyclic group, Y^{a} may be a C1 to C20 divalent linear or cyclic hydrocarbon group, m denotes an integer ranging from 1 to 5, M⁻ may be —SO₃⁻ or —CO₂⁻, and A⁺ may be a cation component.

Exemplary structures of R¹ included in Ra of Formula 1 may be the same as described above.

In Formula 1, Y^{a} may be a C1 to C5 substituted or unsubstituted alkylene group, a C5 to C20 divalent monocyclic or condensed alicyclic hydrocarbon group, or a C5 to C20 divalent monocyclic or condensed aromatic hydrocarbon group.

In an implementation, in Formula 1, Y^{a} may be —(CH₂)ₙ— (in which n is an integer of 1 to 5).

In an implementation, in Formula 1, Y^{a} may be one of the following structures.

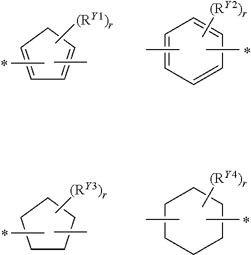

“*” indicates a bonding site, r may be an integer 0 to 2, R^{Y1}, R^{Y2}, R^{Y3}, and R^{Y4} may each independently be a C1 to C10 linear or branched alkyl group, a cyclopropyl group, a cyclopentyl group, or a cyclohexyl group.

In Formula 1, A⁺ may be a sulfonium cation, an iodonium cation, or an ammonium cation. In example embodiments, A+ may include a sulfonium cation represented by Formula 1a, an iodonium cation represented by Formula 1b, or an ammonium cation represented by Formula 1c:

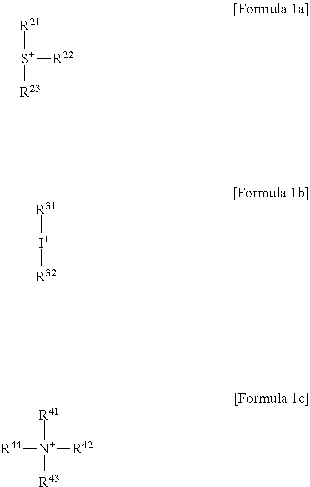

R²¹, R²², R²³, R³¹, R³², R⁴¹, Roz and R⁴³ may independently be, e.g., a C1-C30 hydrocarbon group, which may include a heteroatom, and R44 may be a C1-C30 hydrocarbon group, which may include a heteroatom, or a hydrogen atom. In an implementation, two of R²¹, R²², and R²³ may be bonded together to form a ring with a sulfur atom to which the two selected ones are bonded. In an implementation, two of R⁴¹, R⁴², R⁴³, and R⁴⁴ may be bonded together to form a ring with a nitrogen atom to which the two selected ones are bonded.

In an implementation, in Formulas 1a, 1b, and 1c, each of the hydrocarbon groups included in R²¹, R²², R²³, R³¹, R³², R⁴¹, R⁴², R⁴³ and R⁴⁴ may be a linear, branched, or cyclic hydrocarbon group. In an implementation, the hydrocarbon group included in R²¹, R²², R²³, R³¹, R³², R⁴¹, R⁴², R⁴³, and R⁴⁴ may include an alkyl group, such as methyl, ethyl, n-propyl, isopropyl, n-butyl, and t-butyl; a monovalent saturated cycloaliphatic hydrocarbon group, such as cyclopropyl, cyclopentyl, cyclohexyl, cyclopropylmethyl, 4-methylcyclohexyl, cyclohexylmethyl, norbornyl, and adamantyl; an alkenyl group, such as vinyl, allyl, prophenyl, butenyl, and hexenyl; a monovalent unsaturated cycloaliphatic hydrocarbon group, such as cyclohexenyl; an aryl group, such as phenyl and naphthyl; a heteroaryl group, such as thienyl; or an aralkyl group, such as benzyl, 1-phenylethyl, and 2-phenylethyl.

In an implementation, in R²¹, R²², R²³, R³¹, R³², R⁴¹, R⁴², R⁴³ and R⁴⁴ of Formulae 1a 1b, and 1c, some of hydrogen atoms may be substituted with a heteroatom-containing group, such as oxygen, sulfur, nitrogen, or a halogen. In an implementation, in R²¹, R²², R²³, R³¹, R³², R⁴¹, R⁴², R⁴³ and R⁴⁴ of Formulae 1a, 1b, and 1c, some of carbon atoms may be substituted with a heteroatom-containing group, such as oxygen, sulfur, or nitrogen. In an implementation, each of R²¹, R²², R²³, R³¹, R³², R⁴¹, R⁴², R⁴³, and R⁴⁴ may include a hydroxyl moiety, a cyano moiety, a carbonyl moiety, an ether bond, an ester bond, a sulfonic acid ester bond, a carbonate bond, a lactone ring, a sultone ring, carboxylic anhydride, or a haloalkyl moiety.

In an implementation, the sulfonium cation represented by Formula 1a may include a fluorine atom. In an implementation, in Formula 1, A⁺ may be represented by Formula 1d.

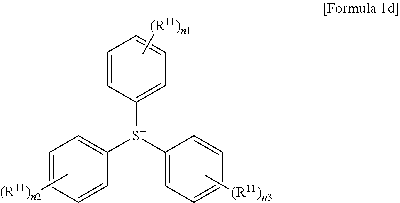

Each R¹¹ may be *—OC(═O)—(CF₂)ₖCF₃, in which k may be an integer of 0 to 10 and * indicates a bonding site. n1, n2, and n3 may each independently be an integer of 0 to 2. In an implementation, at least one of n1, n2, and n3 may be 1 or 2.

In an implementation, in Formula 1, A⁺ may have a structure of Formula 1e.

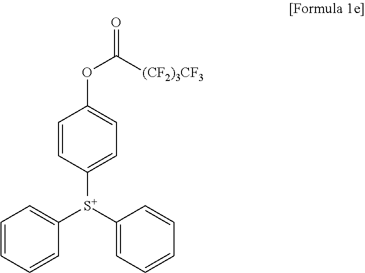

As shown in Formulas 1d and 1e, perfluorinated carbohydrate having a relatively high absorbance may be included in the sulfonium cation, and absorbance may be improved during exposure without suppressing the generation of hydrogen ions due to the dissociation of sulfonium. In an implementation, by applying a photolithography process to a photo-decomposable compound including the sulfonium cation represented by Formula 1d, a difference in solubility in a developer between an exposed area and a non-exposed area of a photoresist film may be larger at the same amount of exposure than when a photo-decomposable compound does not include perfluorinated carbohydrate. Thus, a contrast may be further improved.

In an implementation, the sulfonium cation represented by Formula 1a may not include the fluorine atom. In an implementation, in Formula 1, A⁺ may be, e.g., one of the following structures.

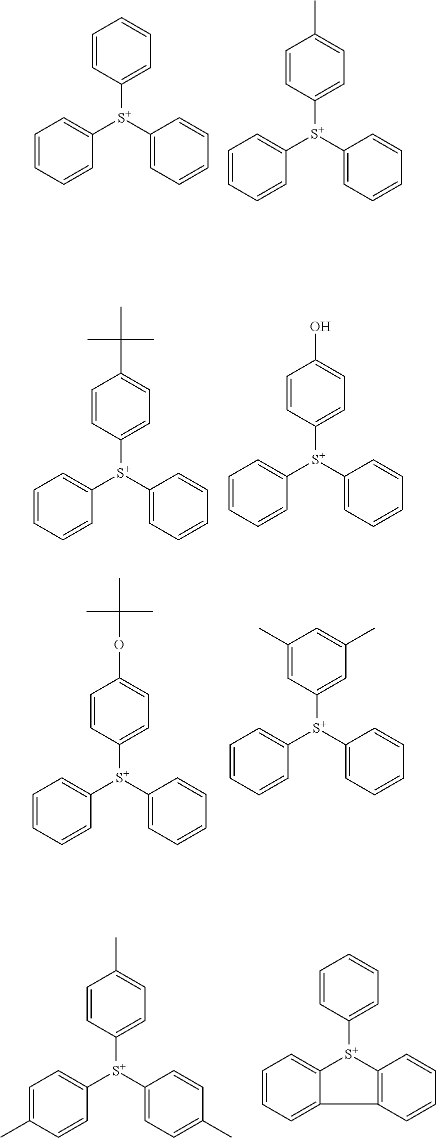

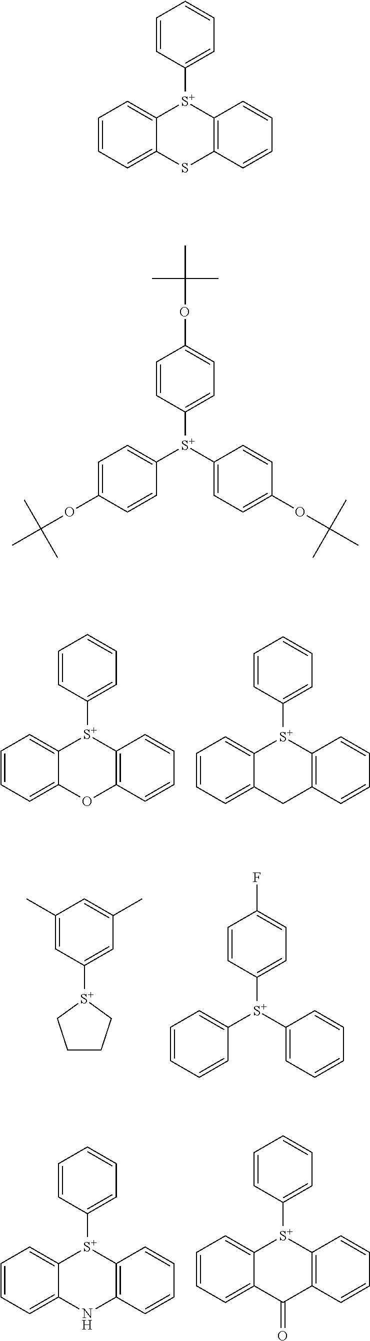

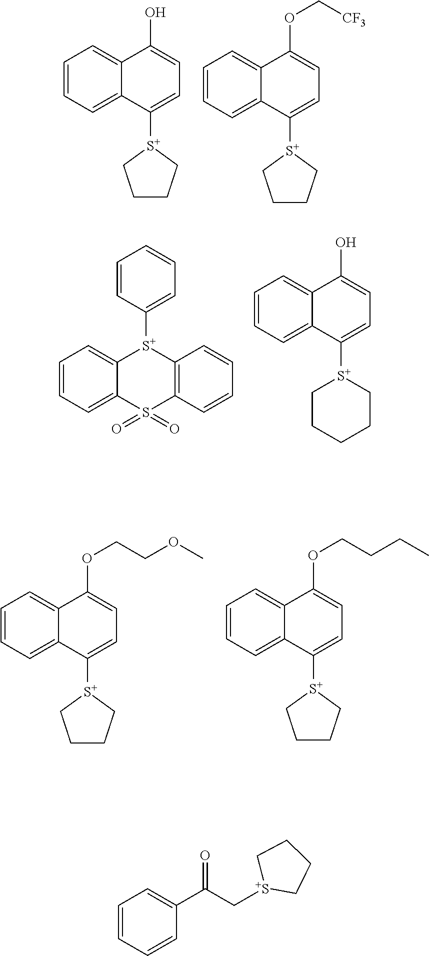

Examples of the iodonium cation represented by Formula 1b may include cations of diphenyliodonium, bis(4-methylphenyl)iodonium, bis(4-ethylphenyl)iodonium, bis(4-(1,1-dimethylethyl)phenyl)iodonium, bis(4-(1,1-dimethylpropyl)phenyl)iodonium, bis(4-tert-butylphenyl)iodonium, bis(4-(1,1-dimethylpropyl)phenyl)iodonium, (4-(1,1-dimethylethoxy)phenyl)phenyliodonium, 4-methoxyphenylphenyliodonium, 4-tert-butoxyphenylphenyli odonium, 4-acryloyloxyphenylphenyliodonium, and 4-(meth)acryloyl oxyphenylphenyliodonium.

Examples of the ammonium cation represented by Formula 1c may include tertiary ammonium cations, such as cations of trimethylammonium, triethylammonium, tributylammonium, and N,N-dimethylanilinium; and quaternary ammonium cations, such as cations of tetramethylammonium, tetraethylammonium, and tetrabutylammonium.

In an implementation, the photo-decomposable compound of Formula 1 may be represented by Formula 1-1:

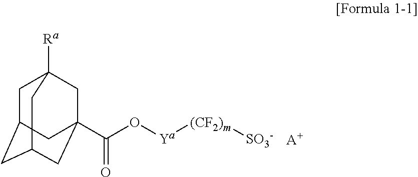

R^{a}, Y^{a}, m, and A⁺ may be the same as described above.

In an implementation, the photo-decomposable compound of Formula 1 may be represented by Formula 1-2:

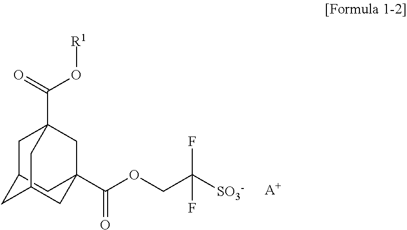

R¹ and A⁺ may be the same as described above.

In an implementation, in the photo-decomposable compound according to the embodiments, the substituent, which decomposes by the action of acid and generates the alkali soluble group, may be connected to the C5 to C40 cyclic hydrocarbon group included in the cation component of the photo-decomposable compound. In an implementation, the photo-decomposable compound may be represented by Formula 2.

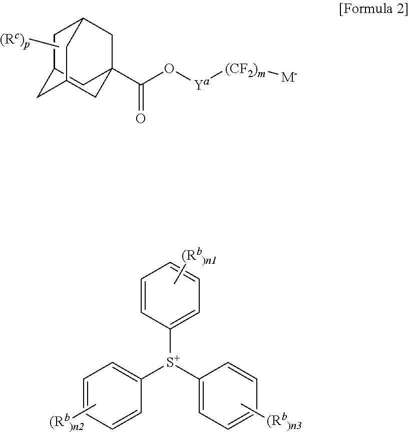

In Formula 2, R^{b} may be a substituent, which decomposes by the action of acid and generates an alkali soluble group, and may be represented by *—C(═O)OR¹. In an implementation, R¹ may be an acid-labile protecting group, which includes substituted or unsubstituted t-butyl or a C3 to C30 substituted or unsubstituted tertiary alicyclic group, Y^{a} may be a C1 to C20 divalent linear or cyclic hydrocarbon group, m may be an integer ranging from 1 to 5, each of n1, n2, and n3 may be an integer ranging from 0 to 2, at least one of n1, n2, and n3 may be 1 or 2, R^{c} may be a C1 to C10 alkyl group, a C1 to C10 alkoxy group, a halogen atom, a C1 to C10 halogenated alkyl group, a hydroxyl group, an unsubstituted C6 to C30 aryl group, or a C6 to C30 aryl group in which some of carbon atoms included in R^{c} may be substituted with a halogen atom or a heteroatom-containing group, p may be an integer ranging from 0 to 2, and M⁻ may be —SO₃⁻ or —CO₂⁻.

Exemplary structures of R¹ included in R^{b} of Formula 2 may be the same as those of R¹ included in R^{a} of Formula 1.

In Formula 2, the halogen atom that may be included in R^{c} may be, e.g., a F atom, a C1 atom, a Br atom, or an I atom. The halogenated alkyl group may include a F atom, a C1 atom, a Br atom, or an I atom. The heteroatom may be an oxygen atom, a sulfur atom, or a nitrogen atom. For example, the heteroatom-containing group may be —O—, —C(═O)—O—, —O—C(═O)—, —C(═O)—, —O—C(═O)—O—, —C(═O)—NH—, —NH—, —S—, —S(═O)₂—, or —S(═O)₂—O—.

In Formula 2, examples of R^{b} may be the same as the above-described examples of R^{a} of Formula 1. In Formula 2, a detailed description of Y^{a}, m, M⁻, and A⁺ may be the same as described above.

In an implementation, the photo-decomposable compound of Formula 2 may be represented by Formula 2-1.

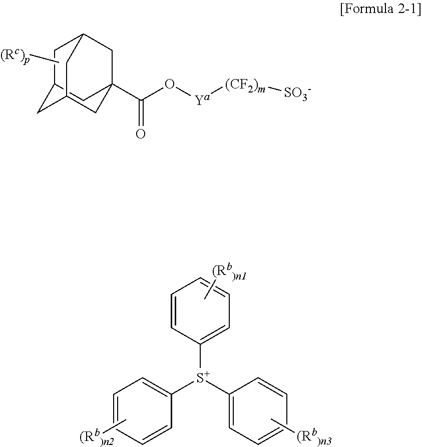

R^{b}, R^{c}, Y^{a}, m, p, n1, n2, and n3 may be the same as described above.

In an implementation, the photo-decomposable compound of Formula 2 may be represented by Formula 2-2.

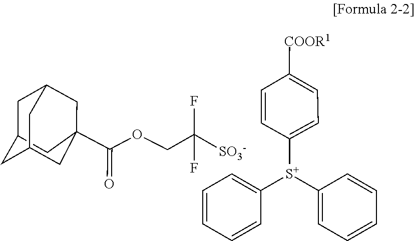

R¹ may be the same as described above.

In an implementation, in the photo-decomposable compound according to the embodiments, the substituent, which decomposes by the action of acid to generate the alkali soluble group, may be connected to each of the adamantyl group included in the anion component of the photo-decomposable compound and the C5 to C40 cyclic hydrocarbon group included in the cation component of the photo-decomposable compound. In an implementation, the photo-decomposable compound may be represented by Formula 3:

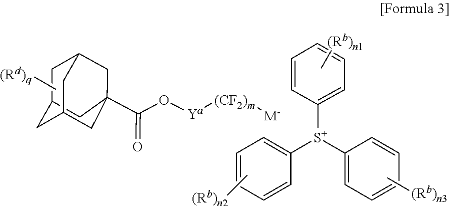

In Formula 3, each of R^{b} and R^{d} may be the substituent, which decomposes by the action of acid and generates an alkali soluble group, and may be represented by *—C(═O)OR¹. In an implementation, R¹ may be an acid-labile protecting group, and may include substituted or unsubstituted t-butyl or a C3 to C30 substituted or unsubstituted tertiary alicyclic group, Y^{a} may be a C1 to C20 divalent linear or cyclic hydrocarbon group, m may be an integer ranging from 1 to 5, each of n1, n2, and n3 may be an integer ranging from 0 to 2, at least one of n1, n2, and n3 may be 1 or 2, q may be 1 or 2, and M⁻ may be —SO₃⁻ or —CO₂⁻.

In Formula 3, R¹ may be the same as described above.

In an implementation, in Formula 3, R^{b} and R^{d} may have different structures. In an implementation, in Formula 3, R^{b} and R^{d} may have the same structure.

In an implementation, the photo-decomposable compound of Formula 3 may be represented by Formula 3-1:

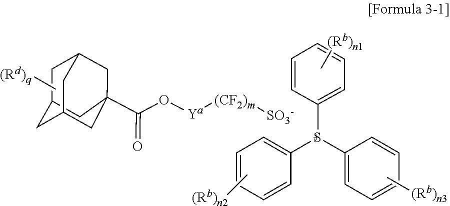

R^{b}, R^{d}, Y^{a}, m, q, n1, n2, and n3 may be the same as described above.

In an implementation, the photo-decomposable compound of Formula 3 may be represented by Formula 3-2

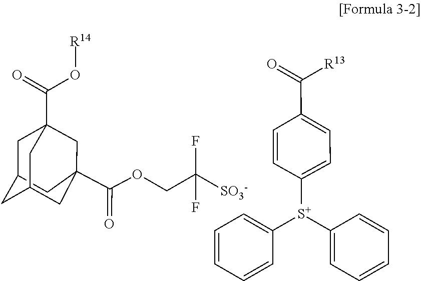

R¹³ and R¹⁴ may be defined the same as R¹³ and R¹⁴ may have different structures or the same structure.

A photoresist composition according to embodiments may include a chemically amplified polymer, a photo-decomposable compound, and a solvent. The photo-decomposable compound may include the photo-decomposable compound according to the above-described embodiment. The photo-decomposable compound may have an anion component including an adamantyl group and a cation component including a C5 to C40 cyclic hydrocarbon group and forming a complex with the anion component. At least one of the adamantyl group included in the anion component and the C5 to C40 cyclic hydrocarbon group included in the cation component may have a substituent, which decomposes by the action of acid and generates an alkali soluble group, and the substituent may include an acid-labile protecting group. A detailed description of the photo-decomposable compound may be the same as given above.

In the photoresist composition according to the embodiments, the photo-decomposable compound may be included in an amount of about 0.1% to about 5.0% by weight, based on a total weight of the chemically amplified polymer.

In the photoresist composition according to the embodiments, the chemically amplified polymer may include a polymer including a repeating unit of which solubility in a developer may be changed by the action of acid. The chemically amplified polymer may be a block copolymer or a random copolymer. In an implementation, the chemically amplified polymer may include positive-type photoresist. The positive-type photoresist may be krypton fluoride (KrF) excimer laser (248 nm) resist, argon fluoride (ArF) excimer laser (193 nm) resist, fluorine (F2) excimer laser (157 nm) resist, or extreme ultraviolet (EUV) (13.5 nm) resist.

In an implementation, the chemically amplified polymer may include a repeating unit, which decomposes by the action of acid and increases solubility in an alkali developer. In other example embodiments, the chemically amplified polymer may include a repeating unit, which decomposes by the action of acid and generates phenolic acid or BrØnsted acid corresponding to the phenolic acid. In an implementation, the chemically amplified polymer may include a first repeating unit, which is derived from hydroxystyrene or derivatives thereof. The derivatives of hydroxystyrene may include hydroxystyrenes in which a hydrogen atom at an a position is substituted with a C1 to C5 alkyl group or a C1 to C5 halogenated alkyl group, and derivatives thereof. For example, the first repeating unit may be derived from 3-hydroxystyrene, 4-hydroxystyrene, 5-hydroxy-2-vinylnaphtalene, or 6-hydroxy vinylnaphtalene.

In an implementation, the chemically amplified polymer may have a structure in which the first repeating unit derived from hydroxystyrene or the hydroxystyrene derivative is copolymerized with at least one second repeating unit having an acid-labile protecting group.

The at least one second repeating unit may include a (meth)acrylate-based polymer. For example, the at least one second repeating unit may include polymethylmethacrylate (PMMA), poly(t-butylmethacrylate), poly(methacrylic acid), poly(norbornylmethacrylate), or a binary or ternary copolymer of repeating units of the (meth)acrylate-based polymers.

In an implementation, the chemically amplified polymer may include a blend of a first polymer having the first repeating unit and a second polymer having the at least one second repeating unit.

The acid-labile group, which may be included in the at least one second repeating unit, may include tert-butoxycarbonyl (t-BOC), isonorbornyl, 2-methyl-2-adamantyl, 2-ethyl-2-adamantyl, 3-tetrahydrofuranyl, 3-oxocyclohexyl, γ-butyllactone-3-yl, mavaloniclactone, γ-butyrolactone-2-yl, 3-methyl-γ-butyrolactone-3-yl, 2-tetrahydropyranyl, 2-tetrahydrofuranyl, 2,3-propylenecarbonate-1-yl, 1-methoxyethyl, 1-ethoxyethyl, 1-(2-methoxyethoxy)ethyl, 1-(2-acetoxyethoxy)ethyl, t-buthoxycarbonylmethyl, methoxymethyl, ethoxymethyl, trimethoxysilyl, or a triethoxysilyl group.

In an implementation, the chemically amplified polymer may further include at least one of a third repeating unit having an acrylate derivative substituent including a hydroxyl group (—OH) and a fourth repeating unit having a protecting group substituted with fluorine.

The chemically amplified polymer may have a weight-average molecular weight of about 1,000 to about 500,000. In the photoresist composition, the chemically amplified polymer may be included in an amount of about 1% to about 25% by weight, based on a total weight of the photoresist composition. Maintaining the content of the chemically amplified polymer at about 1% by weight or greater may help ensure that the photoresist composition may be smoothly coated. Maintaining the content of the chemically amplified polymer at about 25% by weight or less may help ensure that the viscosity of the photoresist composition is not excessively increased, facilitating uniform coating of the photoresist composition.

In an implementation, the photo-decomposable compound included in the photoresist composition according to the embodiments may act as a quenching base that neutralizes acid. In an implementation, the photo-decomposable compound included in the photoresist composition according to the embodiments may generate acid due to exposure.

In an implementation, the photoresist composition according to the embodiments may further include a photoacid generator (PAG), which generates acid due to exposure.

The PAG may include a material having a different chemical structural formula from that of the photo-decomposable compound. In an implementation, the PAG may generate acid when exposed to any one of a KrF excimer laser (248 nm), an ArF excimer laser (193 nm), an F2 excimer laser (157 nm), and an EUV laser (13.5 nm). The PAG may include a material that generates a relatively strong acid having a pKa of about −20 or more and less than about 1 due to exposure. The PAG may include, e.g., triarylsulfonium salts, diaryliodonium salts, sulfonates, or a mixture thereof. In an implementation, the PAG may include triphenylsulfonium triflate, triphenylsulfonium antimonate, diphenyliodonium triflate, diphenyliodonium antimonate, methoxydiphenyliodonium triflate, di-t-butyldiphenyliodonium triflate, 2,6-dinitrobenzyl sulfonates, pyrogallol tris(alkylsulfonates), N-hydroxysuccinimide triflate, norbornene-dicarboximide-triflate, triphenylsulfonium nonaflate, diphenyliodonium nonaflate, methoxydiphenyliodonium nonaflate, di-t-butyldiphenyliodonium nonaflate, N-hydroxysuccinimide nonaflate, norbornene-dicarboximide-nonaflate, triphenylsulfonium perfluorobutanesulfonate, triphenylsulfonium perfluorooctanesulfonate (PFOS), diphenyliodonium PFOS, methoxydiphenyliodonium PFOS, di-t-butyldiphenyliodonium triflate, N-hydroxysuccinimide PFOS, norbornene-dicarboximide PFOS, or a mixture thereof.

In the photoresist composition according to the embodiments, the PAG may be included in an amount of about 0.1% to about 5.0% by weight, based on a total weight of the chemically amplified polymer.

In an implementation, the photoresist composition according to the embodiments may further include a basic quencher.

When acid generated by the photo-decomposable compound or the PAG, which is included in the photoresist composition according to the embodiments, diffuses into a non-exposed area of a photoresist film, the basic quencher may be a compound capable of trapping the acid in the non-exposed area of the photoresist film. Because the basic quencher is included in the photoresist composition according to the embodiments, a diffusion rate of the acid may be inhibited.

In an implementation, the basic quencher may include primary aliphatic amine, secondary aliphatic amine, tertiary aliphatic amine, aromatic amine, heterocyclic amine, a nitrogen-containing compound having a carboxyl group, a nitrogen-containing compound having a sulfonyl group, a nitrogen-containing compound having a hydroxyl group, a nitrogen-containing compound having a hydroxyphenyl group, an alcoholic nitrogen-containing compound, amides, imides, carbamates, or ammonium salts. In an implementation, the basic quencher may include triethanol amine, triethyl amine, tributyl amine, tripropyl amine, hexamethyl disilazan, aniline, N-methylaniline, N-ethylaniline, N-propylaniline, N,N-dimethylaniline, N,N-bis(hydroxyethyl)aniline, 2-methylaniline, 3-methylaniline, 4-methylaniline, ethylaniline, propylaniline, dimethylaniline, 2,6-diisopropylaniline, trimethylaniline, 2-nitroaniline, 3-nitroaniline, 4-nitroaniline, 2,4-dinitroaniline, 2,6-dinitroaniline, 3,5-dinitroaniline, N,N-dimethyltoluidine, or a combination thereof.

In an implementation, the basic quencher may include a photo-decomposable base. The photo-decomposable base may include a compound, which generates acid due to exposure and neutralizes the acid before exposure or in an unexposed state. The photo-decomposable base may lose ability to trap acid when decomposed due to exposure. In an implementation, when a partial region of a photoresist film that is formed using a chemically amplified photoresist composition including the basic quencher including the photo-decomposable base is exposed, the photo-decomposable base may lose alkalinity in an exposed area of the photoresist film, while the photo-decomposable base may trap acid in a non-exposed area of the photoresist film to inhibit the diffusion of the acid from the exposed area into the non-exposed area.

The photo-decomposable base may include a carboxylate or sulfonate salt of a photo-decomposable cation. In an implementation, the photo-decomposable cation may form a complex with an anion of C1 to C20 carboxylic acid. The carboxylic acid may be, e.g., formic acid, acetic acid, propionic acid, tartaric acid, succinic acid, cyclohexylcarboxylic acid, benzoic acid, or salicylic acid.

In the photoresist composition according to the embodiments, the basic quencher may be included in an amount of about 0.01% to about 0.5% by weight, based on a total weight of the chemically amplified polymer.

In the photoresist composition according to the embodiments, the solvent may include an organic solvent. In an implementation, the solvent may include at least one of ether, alcohol, glycol ether, an aromatic hydrocarbon compound, ketone, and ester. In an implementation, the solvent may be selected from ethyleneglycolmonomethylether, ethyleneglycolmonoethylether, methylcellosolveacetate, ethylcellosolveacetate, diethyleneglycolmonomethylether, diethyleneglycolmonoethylether, propyleneglycol, propyleneglycolmonomethylether, propyleneglycolmonomethyletheracetate, propyleneglycolmonoethylether, propyleneglycolmonoethyletheracetate, propyleneglycolpropyletheracetate, propyleneglycolmonobutylether, propyleneglycolmonobutyletheracetate, toluene, xylene, methylethyl ketone, cyclopentanone, cyclohexanone, 2-hydroxypropionate ethyl, 2-hydroxy-2-methylpropionate ethyl, ethyl ethoxyacetate, ethyl hydroxyacetate, 2-hydroxy-3-methylbutanoate methyl, 3-methoxypropionate methyl, 3-methoxypropionate ethyl, 3-ethoxypropionate ethyl, 3-ethoxypropionate methyl, methyl pyruvate, ethyl pyruvate, ethyl acetate, butyl acetate, ethyl lactoate, and butyl lactoate. The solvents may be used alone or in combination of at least two thereof. In an implementation, the amount of the solvent in the photoresist composition may be adjusted so that a solid content of the photoresist composition may range from about 3% to about 20% by weight.

In an implementation, the photoresist composition according to the embodiments may further include a surfactant.

The surfactant may include, e.g., fluoroalkylbenzenesulfonate, fluoroalkyl carboxylate, fluoroalkylpolyoxyethyleneether, fluoroalkylammonium iodide, fluoroalkylbetaine, fluoroalkylsulfonate, diglycerin tetrakis (fluoroalkyl polyoxyethyleneether), fluoroalkyl trimethylammonium salt, fluoroalkylaminosulfonate, polyoxyethylenenonylphenylether, polyoxyethyleneoctylphenylether, polyoxyethylenealkylether, polyoxyethylenelaurylether, polyoxyethylene oleylether, polyoxyethylene tridecylether, polyoxyethylene cetylether, polyoxyethylene stearylether, polyoxyethylenelaurate, polyoxyethylene oleate, polyoxyethylenestearate, polyoxyethylenelaurylamine, sorbitanlaurate, sorbitanpalmitate, sorbitanstearate, sorbitan oleate, sorbitan fatty acid ester, polyoxyethylene sorbitanlaurate, polyoxyethylene sorbitanpalmitate, polyoxyethylenesorbitanstearate, polyoxyethylenesorbitan oleate, polyoxyethylene naphthylether, alkylbenzenesulfonate, or alkyldiphenyletherdisulfonate. The surfactant may be included in an amount of about 0.001% to about 0.1% by weight, based on the total weight of the chemically amplified polymer.

In the photoresist composition according to the embodiments, acid may be generated from the photo-decomposable compound due to exposure. The generated acid may act on a protecting group of the chemically amplified polymer to deprotect the chemically amplified polymer. Thus, the deprotected polymer may be changed into an alkali soluble group.

A vast amount of research has been conducted into an EUV lithography technique incorporating an exposure process using EUV light having a wavelength of about 13.5 nm as an advanced technique for superseding a lithography process using a KrF excimer laser (248 nm) and an ArF excimer laser (193 nm). An EUV lithography process may be based on a different action mechanism from the lithography process using the KrF excimer laser and the ArF excimer laser. The entire EUV lithography process may be performed in vacuum. Because an EUV lithography system lacks power required for a light source to irradiate laser light, there may be limit to sufficiently increasing a dose to generate a required amount of acid from a PAG, from among components of a photoresist composition, during an exposure process. Thus, when an EUV lithography process is performed using a typical photoresist composition including only a PAG, acid generation efficiency and an exposure speed may be reduced due to a relatively low dose provided by a light source of the EUV lithography system. Accordingly, it may be difficult to obtain a desired exposure sensitivity.

The photoresist composition according to the embodiments may include the photo-decomposable compound according to the embodiments, and the photo-decomposable compound may generate acid due to exposure and act as a quenching base that neutralizes the acid. Accordingly, when a photoresist film formed using the photoresist composition is exposed, acid may be generated from the photo-decomposable compound in an exposed area of the photoresist film. Also, the photo-decomposable compound may act as a quenching base to neutralize an acid in a non-exposed area of the photoresist film. Thus, a difference in acidity between the exposed area and the non-exposed area of the photoresist film may be increased.

In an implementation, in the photo-decomposable compound included in the photoresist composition according to the embodiments, at least one of the adamantyl group included in the anion component of the photo-decomposable compound and the C5 to C40 cyclic hydrocarbon group included in the cation component of the photo-decomposable compound may have a substituent, which decomposes by the action of acid and generates an alkali soluble group, and the substituent may include an acid-labile protecting group. In a photolithography process using the photoresist composition including the photo-decomposable compound according to the embodiments, when a partial region of a photoresist film obtained using the photoresist composition is exposed, acid having a relatively bulky structure may be generated from the photo-decomposable compound in an exposed area of the photoresist film, and thus, a distance by which the acid diffuses may be relatively small. In an implementation, a difference in acidity between the exposed area and a non-exposed area of the photoresist film may be increased, and a difference in solubility in a developer between the exposed area and the non-exposed area of the photoresist film may be increased and a contrast may be increased. Accordingly, a line edge roughness (LER) and a line width roughness (LWR) may be reduced in a photoresist pattern obtained by developing the exposed photoresist film, and thus, a high pattern fidelity may be achieved. In addition, by manufacturing an integrated circuit (IC) device using a photoresist composition according to an embodiment, the dimensional precision of a pattern required for the IC device may be improved, and the productivity of a process of manufacturing the IC device may be increased.

Hereinafter, a method of manufacturing an IC device, according to an example embodiment will be described.

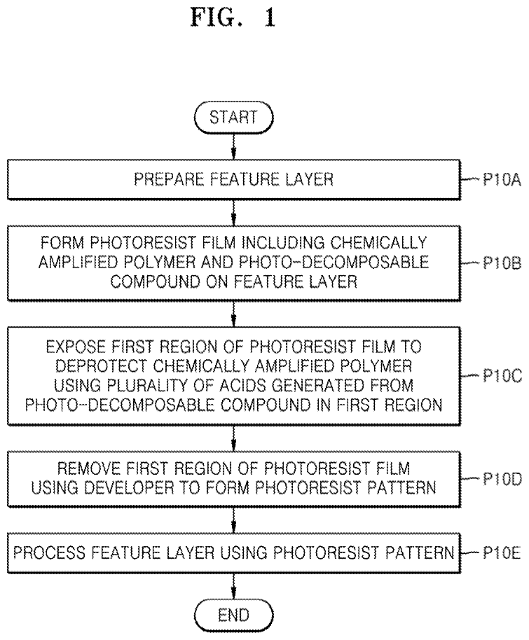

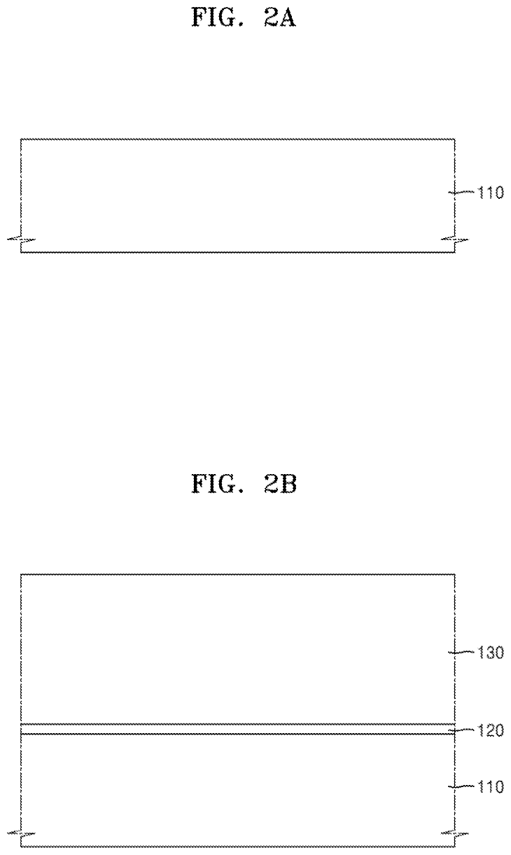

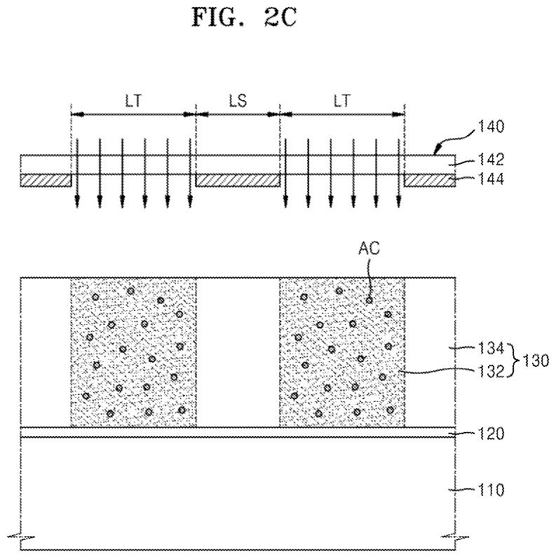

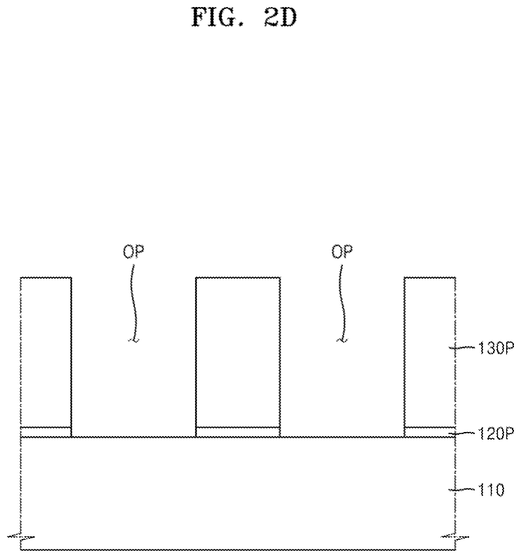

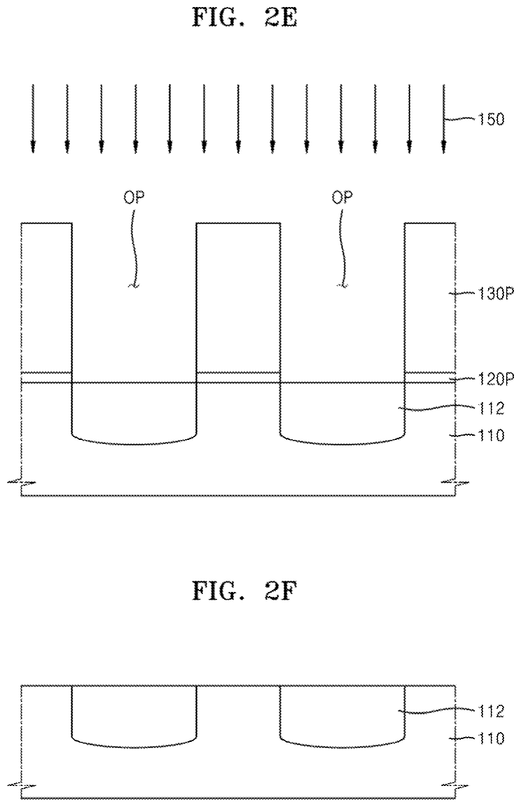

FIG. 1 is a flowchart of a method of manufacturing an IC device, according to embodiments. FIGS. 2A to 2F are cross-sectional views of stages in a method of manufacturing an IC device, according to embodiments.

Referring to FIGS. 1 and 2A, in process P10A of FIG. 1, a feature layer 110 may be prepared.

In an implementation, the feature layer 110 may include a semiconductor substrate. In an implementation, the feature layer 110 may include a semiconductor, such as silicon (Si) or germanium (Ge), or a compound semiconductor, such as silicon germanium (SiGe), silicon carbide (SiC), gallium arsenide (GaAs), indium arsenide (InAs), or indium phosphide (InP). In an implementation, the feature layer 110 may include a conductive film, a dielectric film, an insulating film, or a combination thereof, which is formed on the semiconductor substrate. For example, the feature layer 110 may include a metal, an alloy, a metal carbide, a metal nitride, a metal oxynitride, a metal oxycarbide, a semiconductor, polysilicon, oxide, nitride, oxynitride, or a combination thereof.

Referring to FIGS. 1 and 2B, in process P10B, a chemically amplified polymer and a photoresist film 130 including a photo-decomposable compound according to an embodiment may be formed on the feature layer 110.

In an implementation, the photoresist film 130 may further include at least one of a PAG and a basic quencher. A detailed description of the PAG and the basic quencher may be the same as given above.

In an implementation, before the photoresist film 130 is formed on the feature layer 110, a developable bottom anti-reflective coating (DBARC) film 120 may be formed on the feature layer 110, and the photoresist film 130 may be formed on the DBARC film 120. The DBARC film 120 may control diffuse reflection of light from a light source used during an exposure process for manufacturing an IC device or absorb reflected light from the feature layer 110 located thereunder. In an implementation, the DBARC film 120 may include an organic anti-reflective coating (ARC) material for a KrF excimer laser, an ArF excimer laser, or any other light source. In an implementation, the DBARC film 120 may include an organic component having a light-absorbing structure. The light-absorbing structure may include, for example, at least one benzene ring or a hydrocarbon compound in which benzene rings are fused. The DBARC film 120 may be formed to a thickness of about 20 nm to about 100 nm.

To form the photoresist film 130, the DBARC film 120 may be coated with a photoresist composition according to an embodiment, and an annealing process may be performed. The coating process may be performed using, e.g., a spin coating process, a spray coating process, and a dip coating process. The process of annealing the photoresist composition may be performed at a temperature of about 80° C. to about 150° C. for about 10 seconds to about 100 seconds. A thickness of the photoresist film 130 may be several times to several hundred times a thickness of the DBARC film 120. The photoresist film 130 may be formed to a thickness of about 100 nm to about 6 μm.

Referring to FIGS. 1 and 2C, in process P10C, a first area 132, which is a portion of the photoresist film 130, may be exposed to generate a plurality of acids AC in the first area 132 of the photoresist film 130. The plurality of acids AC may be derived from the photo-decomposable compound. The chemically amplified polymer may be deprotected by the plurality of acids AC in the first region 132 of the photoresist film 130.

In an implementation, when the photoresist film 130 further includes the PAG, additional acids may be further generated from the PAG in the first area 132 of the photoresist film 130, and the chemically amplified polymer may be deprotected due to the additional acids.

To expose the first area 132 of the photoresist film 130, a photomask 140 having a plurality of light-shielding areas LS and a plurality of light-transmitting areas LT may be aligned at a predetermined position on the photoresist film 130, and the first area 132 of the photoresist film 130 may be exposed through the plurality of light-transmitting areas LT of the photomask 140. The first area 132 of the photoresist film 130 may be exposed using a KrF excimer laser (248 nm), an ArF excimer laser (193 nm), a F2 excimer laser (157 nm), or an EUV laser (13.5 nm).

The photomask 140 may include a transparent substrate 142 and a plurality of light-shielding patterns 144 formed in the plurality of light-shielding areas LS on the transparent substrate 142. The transparent substrate 142 may include quartz. The plurality of light-shielding patterns 144 may include chromium (Cr). The plurality of light-transmitting areas LT may be defined by the plurality of light-shielding patterns 144.

In an implementation, an annealing process may be performed to diffuse the plurality of acids AC in the first area 132 of the photoresist film 130. In an implementation, the resultant structure, which is obtained directly after the first area 132 of the photoresist film 130 is exposed in process P10C of FIG. 1, may be annealed at a temperature of about 50° C. to about 150° C. In an implementation, at least some of the plurality of acids AC may be diffused in the first area 132 of the photoresist film 130 so that the plurality of acids AC may be relatively uniformly distributed in the first area 132 of the photoresist film 130. The annealing process may be performed for about 10 seconds to about 100 seconds. In an implementation, the annealing process may be performed at a temperature of about 100° C. for about 60 seconds.

In an implementation, an additional annealing process may not be performed to diffuse the plurality of acids AC in the first area 132 of the photoresist film 130. In this case, in process P10C of FIG. 1, during the exposing of the first area 132 of the photoresist film 130, the plurality of acids AC may be diffused in the first area 132 of the photoresist film 130 without an additional annealing process.

As a result of the diffusion of the plurality of acids AC in the first area 132 of the photoresist film 130, an acid-labile group may be deprotected from a chemically amplified polymer included in the photoresist film 130 in the first area 132 of the photoresist film 130, and thus, the first area 132 of the photoresist film 130 may be changed to a state in which the first area 132 may be easily dissolved in an alkali developer.

In the first area 132 that is an exposed area, the photo-decomposable compound included in the photoresist film 130 may decompose due to exposure and may not act as a quenching base for neutralizing the plurality of acids AC after the plurality of acids AC are generated. In contrast, because light is not transmitted to the photo-decomposable compound, which is included in the photoresist film 130, in a second area 134 that is a non-exposed area of the photoresist film 130, acid may not be generated from the photo-decomposable compound. Thus, a reaction in which the acid-labile group is deprotected from the chemically amplified polymer may not occur in the second area 134 of the photoresist film 130. Also, the photo-decomposable compound included in the second area 134, which is the non-exposed area of the photoresist film 130, may not be decomposed. In an implementation, in the second area 134 that is the non-exposed area, the photo-decomposable compound included in the photoresist film 130 may act as a quenching base to neutralize acids that have been undesirably diffused from the first area 132 into the second area 134.

As described above, the plurality of acids AC, which are generated from the photo-decomposable compound, may be present in the first area 132 that is the exposed area. The photo-decomposable compound serving as the quenching base may be present in an undecomposed state in the second area 134 that is the non-exposed area. Thus, a difference in acidity between the first area 132, which is the exposed area, and the second area 134, which is the non-exposed area, may be increased. Accordingly, a difference in solubility in a developer between the exposed area and the non-exposed area of the photoresist film 130 may be increased. As a result, a pattern having a low LER or a low LWR may be obtained in a final pattern, which is to be formed in a subsequent process.

Referring to FIGS. 1 and 2D, in process P10D, the photoresist film 130 may be developed using an alkali developer to remove the first area 132 from the photoresist film 130. As a result, a photoresist pattern 130P including the second area 134, which is the non-exposed area, may be formed.

The photoresist pattern 130P may include a plurality of openings OP. A portion of the DBARC film 120, which is exposed through the plurality of openings OP, may be removed to form a DBARC pattern 120P.

The alkali developer may include 2.38% by weight of a tetramethylammonium hydroxide (TMAH) solution. Because the chemically amplified polymer is deprotected by the plurality of acids AC in the first area 132 of the photoresist film 130 in the resultant product of FIG. 2C, the first area 132 may be cleanly removed during the developing of the photoresist film 130 by using the alkali developer. Accordingly, after the photoresist film 130 is developed, residue defects, such as a footing phenomenon, may not occur, and the photoresist pattern 130P may obtain a vertical sidewall profile. As described above, by improving a profile of the photoresist pattern 130P, when the feature layer 110 is processed using the photoresist pattern 130P, a critical dimension (CD) of an intended processing region may be precisely controlled in the feature layer 110.

Referring to FIGS. 1 and 2E, in process P10E, the feature layer 110 may be processed using the photoresist pattern 130P.

In an implementation, various processes including a process of implanting impurity ions into the feature layer 110 through the plurality of openings OP of the photoresist pattern 130P, a process of etching the feature layer 110 through the plurality of openings OP, a process of forming an additional film on the feature layer 110 through the plurality of openings OP, and a process of modifying a portion of the feature layer 110 through the plurality of openings OP may be performed.

FIG. 2E illustrates an ion implantation process as an example of processing the feature layer 110 exposed through the plurality of openings OP. As shown in FIG. 2E, impurity ions 150 may be implanted into the feature layer 110 through the plurality of openings OP, thereby forming a plurality of wells 112 in the feature layer 110. Each of the plurality of wells 112 may include an impurity region into which the impurity ions 150 are implanted. The impurity ions 150 may be an n-type dopant or a p-type dopant.

Referring to FIG. 2F, the photoresist pattern 130P and the DBARC pattern 120P, which remain on the feature layer 110, may be removed from the resultant structure of FIG. 2E. The photoresist pattern 130P and the DBARC pattern 120P may be removed using an ashing process and a strip process.

In the method of manufacturing the photoresist film 130 including the photo-decomposable compound according to the embodiments described with reference to FIGS. 1 and 2A to 2F, a difference in acidity between the exposed area and the non-exposed area may be increased to increase solubility in the developer between the exposed area and the non-exposed area. Thus, an LER and an LWR may be reduced in the photoresist pattern 130P obtained from the photoresist film 130 to provide a high pattern fidelity. Accordingly, when a subsequent process is performed on the feature layer 110 using the photoresist pattern 130P, a dimensional precision may be improved by precisely controlling critical dimensions of processing regions or patterns to be formed in the feature layer 110.

Hereinafter, Synthesis examples of a photo-decomposable compound according to embodiments will be described. The following Synthesis examples are provided to explain a process of synthesizing a photo-decomposable compound according to embodiments, but the scope of the embodiments is not limited thereto.

### Synthesis Example 1

Synthesis of Photo-Decomposable Compound of Formula 4

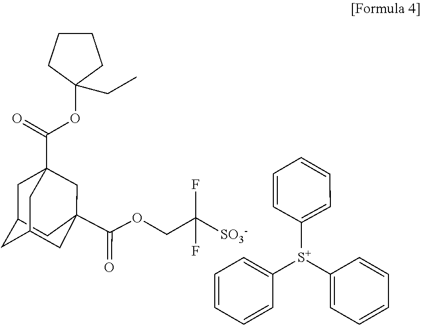

The compound (triphenylsulfonium 2-(((1r,3s,5R,7S)-3-(((1-ethylcyclopentyl)oxy)carbonyl)adamantane-1-carbonyl)oxy)-1,1-difluoroethane-1-sulfonate) of Formula 4 was synthesized according to sequential synthesis processes of the following Chemical equations 1a, 1b, 1c, and 1d.

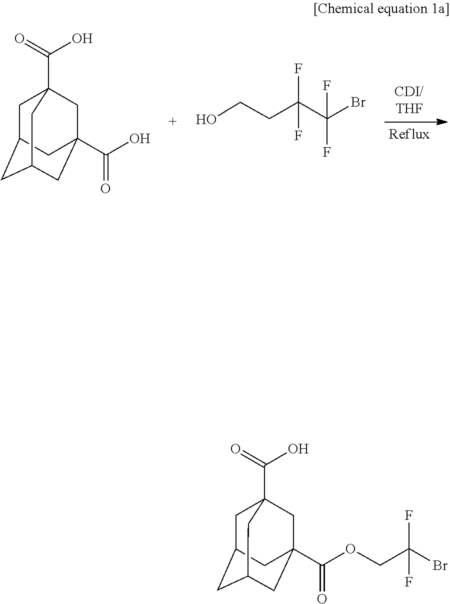 [SMILES: O=C(O)C12CC3CC(C1)CC(C(=O)O)(C3)C2 ; OCCC(F)(F)C(F)(F)Br ; O=C(O)C12CC3CC(C1)CC(C(=O)OCC(F)(F)Br)(C3)C2]

A synthesis process shown in Chemical equation 1a will now be described in detail. 9.4 g (42 mmol) of 4-bromo-3,3,4,4-tetrafluorobutan-1-ol was put in a 250-mL flask, and the 250-mL flask was filled with 100 mL of a dehydrated tetrahydrofuran (THF) solvent under an N₂ atmosphere. A solution in which 7.5 g (46 mmol) of 1′,1′-carbodiimidazole (CDI) was dissolved in 20 mL of dehydrated THF was slowly added dropwise to the obtained solution at ambient temperature. The resultant solution was stirred for 3 hours, and 9.5 g (42 mmol) of adamantane-1,3-dicarboxylic acid was then slowly added dropwise while heating and refluxing the same. The resultant product was refluxed for 12 hours and cooled to ambient temperature, and layer separation was caused in a separatory funnel by putting ethyl acetate and water therein. An organic layer (i.e., an ethyl acetate layer) at an upper position was washed with distilled water three times, dried with dehydrated magnesium sulfate, and filtered. The resultant product was desolventized using a rotary evaporator to obtain 7.8 g of a desired product.

¹H NMR (DMSO, 300 MHz): δ 12.08 (s, 1H), 4.90 (t, 2H), 1.1˜2.02 (m, 12H)

A synthesis process shown in Chemical equation 1b was performed using the compound obtained in Chemical equation 1a.

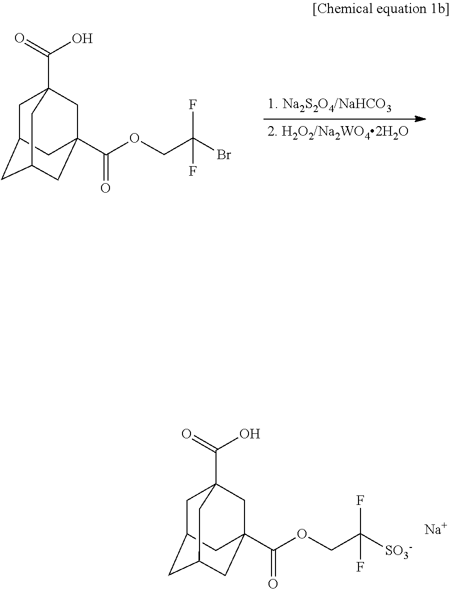

The synthesis process shown in Chemical equation 1b will now be described in further detail. The product obtained in Chemical equation 1a was dissolved in 100 mL of acetonitrile and 100 mL of distilled water in a 500-mL flask. 17.7 g (102 mmol) of sodium hydrosulfite and 12.9 g (153 mmol) of sodium bicarbonate were put in the 500-mL flask and stirred while heating the same at a temperature of 60° C. for 20 hours. Thereafter, the resultant product was cooled to ambient temperature, and an organic layer was separated and transferred to a 500-mL flask. Afterwards, 100 mL of distilled water, 8.7 g (76 mmol) of 30% hydrogen peroxide, and 40 mg (0.12 mmol) of sodium tungstate dihydrate were put in the 500-mL flask and stirred at room temperature for 6 hours. After a reaction was completed, 17.4 g (100 mmol) of sodium hydrosulfite was slowly added to the reacted solution to cause a reaction. Thereafter, the remaining hydrogen peroxide was removed by reduction, and a sodium chloride aqueous solution was put in the 500-mL flask to cause layer separation of an organic layer from an aqueous layer. 300 mL of diethyl ether was added to the resultant product and stirred to separate an organic layer, which was at an upper position, from the resultant product. The separated organic layer was dried with dehydrated magnesium sulfate and filtered, and the resultant product was then desolventized using a rotary evaporator. A synthesis process shown in Chemical equation 1c was then performed without an additional purification process.

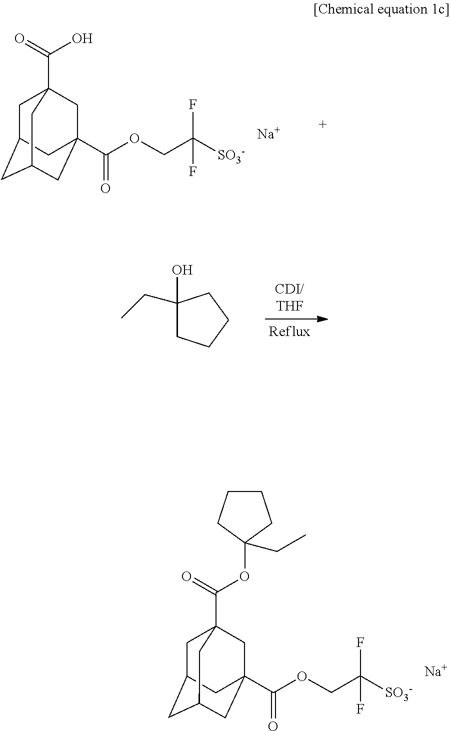

The synthesis process shown in Chemical equation 1c will now be described in detail. 1.7 g (15 mmol) of 1-ethylcyclopentan-1-ol was put in a 250-mL flask, and the 250-mL flask was filled with 100 mL of a dehydrated THF solvent under an N₂ atmosphere. A solution in which 2.25 g (12.6 mmol) of CDI was dissolved in 20 mL of dehydrated THF was slowly added dropwise to the obtained solution at ambient temperature. The resultant product was stirred for three hours, and 4.9 g (12.6 mmol) of the product (sodium 2-(((1s,3r,5R,7S)-3-carboxyadamantane-1-carbonyl)oxy)-1,1-difluoroethane-1-sulfonate) obtained in Chemical equation 1b was then slowly added dropwise while heating and refluxing the same. Thereafter, the resultant product was refluxed for 12 hours and cooled to ambient temperature, and layer separation was caused in a separatory funnel by putting ethyl acetate and water therein. An organic layer (i.e., an ethyl acetate layer) at an upper position was washed with distilled water three times, dried with dehydrated magnesium sulfate, and filtered. The resultant product was desolventized using a rotary evaporator. After that, 4.2 g of a product was separated using a recrystallization process.

¹H NMR (DMSO, 300 MHz): δ 4.95 (t, 2H), 2.15˜1.46 (m, 21H), 1.1 (m, 3H), 0.9 (t, 3H)

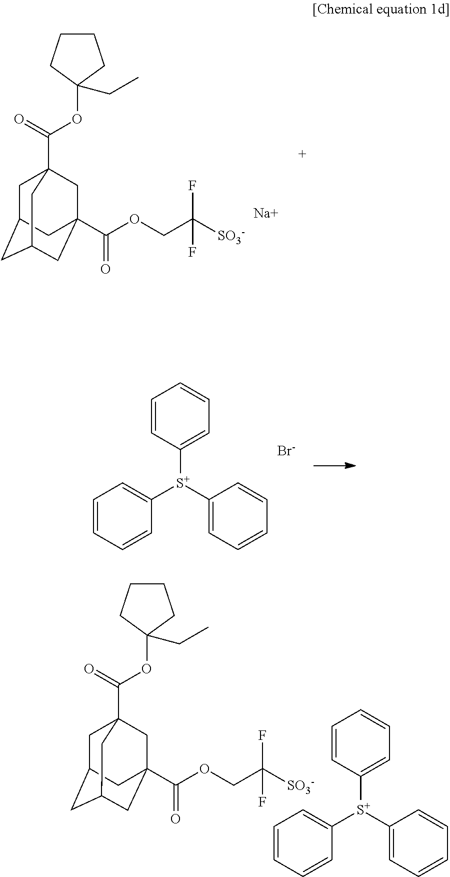

A synthesis process shown in Chemical equation 1d will now be described in detail. 4.2 g (8.6 mmol) of the product (sodium 2-(((1r,3s,5R,7S)-3-(((1-ethylcyclopentyl)oxy)carbonyl)adamantane-1-carbonyl)oxy)-1,1-difluoroethane-1-sulfonate) obtained in Chemical equation 1c, 50 mL of dichloromethane, and 50 mL of distilled water were put in a 250-mL flask, and 3.4 g (10 mmol) of triphenylsulfonium bromide was added to the 250-mL flask and stirred for 20 hours at ambient temperature. An organic layer (i.e., a dichloromethane layer) at a lower position was separated and washed with 100 mL of distilled water three times. Thereafter, the organic layer was dried with dehydrated magnesium sulfate and filtered. The resultant product was desolventized using a rotary evaporator, distilled, and then dissolved again in 20 mL of dichloromethane. 40 mL of diethyl ether was put and cooled to obtain 17.8 g of the compound (triphenylsulfonium 2-(((1r,3s,5R,7S)-3-(((1-ethylcyclopentyl)oxy)carbonyl)adamantane-1-carbonyl)oxy)-1,1-difluoroethane-1-sulfonate) of Formula 4 (yield 93%).

¹H NMR (DMSO, 300 MHz): δ 7.3 (m, 15H), 4.96 (t, 2H), 2.1˜1.76 (m, 2H), 2.2˜1.0 (m, 23H), 0.90 (t, 3H)

### Synthesis Example 2

Synthesis of Photo-Decomposable Compound of Formula 5

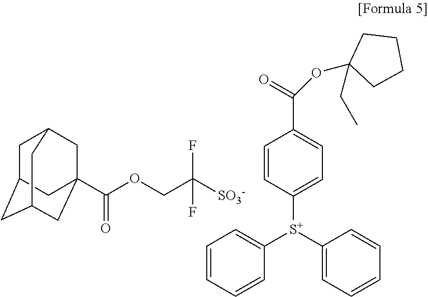

The compound ((4-(((1-ethylcyclopentyl)oxy)carbonyl)phenyl)diphenylsulfonium 2-(((3r,5r,7r)-adamantane-1-carbonyl)oxy)-1,1-difluoroethane-1-sulfonate) of Formula 5 was synthesized according to sequential synthesis processes of the following Chemical equations 2a, 2b, 2c, and 2d.

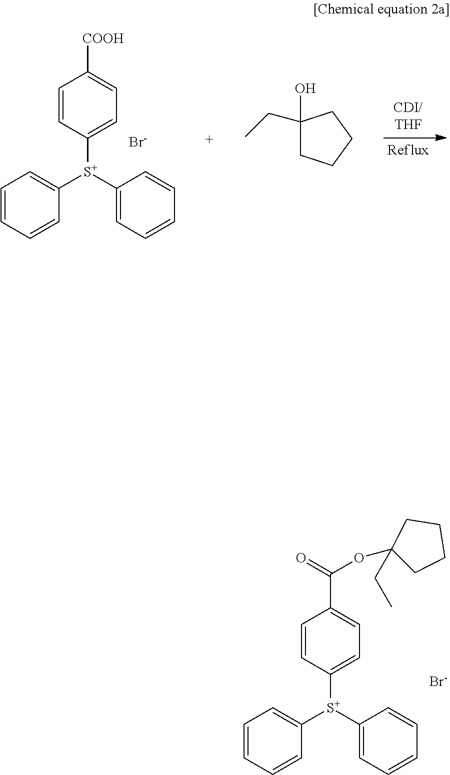

A synthesis process shown in Chemical equation 2a will now be described in further detail. 5.7 g (50 mmol) of 1-ethylcyclopentan-1-ol was put in a 250-mL flask, and the 250-mL flask was filled with 100 mL of a dehydrated THF solvent under an N₂ atmosphere. A solution in which 8.4 g (52 mmol) of CDI was dissolved in 20 mL of dehydrated THF was slowly added dropwise to the obtained solution at ambient temperature. The resultant product was stirred for 3 hours, and 19.4 g (50 mmol) of (4-carboxyphenyl)diphenylsulfonium bromide was then slowly added dropwise while heating and refluxing the same. The resultant product was refluxed for 12 hours and cooled to ambient temperature, and layer separation was caused in a separatory funnel by putting ethyl acetate and water therein. An ethyl acetate layer was washed with a sodium methoxide solution three times. Thereafter, an organic layer (i.e., an ethyl acetate layer) at an upper position was washed with distilled water three times, dried with dehydrated magnesium sulfate, and filtered. The resultant product was desolventized using a rotary evaporator. The resultant product was purified using a recrystallization process to obtain 16.9 g of a desired product.

¹H NMR (DMSO, 300 MHz): δ 8.7 (q, 2H), 7.5 (m, 12H), 2.0˜1.5 (m, 10H), 0.94 (t, 3H)

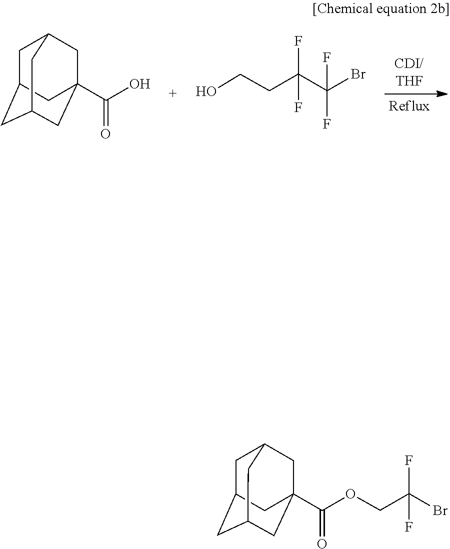 [SMILES: O=C(O)C12CC3CC(CC(C3)C1)C2 ; OCCC(F)(F)C(F)(F)Br ; O=C(OCC(F)(F)Br)C12CC3CC(CC(C3)C1)C2]

A synthesis process shown in Chemical equation 2b will be described in further detail. 11.2 g (50 mmol) of 4-bromo-3,3,4,4-tetrafluorobutan-1-ol was put in a 250-mL flask, and the 250-mL was filled with 100 mL of a dehydrated tetrahydrofuran (THF) solvent under an N₂ atmosphere. A solution in which 8.4 g (52 mmol) of CDI was dissolved in 20 mL of dehydrated THF was slowly added dropwise to the obtained solution at ambient temperature. The resultant solution was stirred for 3 hours, and 9 g (50 mmol) of (3r,5r,7r)-adamantane-1-carboxylic acid was then slowly added dropwise while heating and refluxing the same. The resultant product was refluxed for 12 hours and cooled to ambient temperature, and layer separation was caused in a separatory funnel by putting ethyl acetate and water therein. An ethyl acetate layer was washed with a sodium methoxide solution three times. Thereafter, an organic layer (i.e., an ethyl acetate layer) at an upper position was washed with distilled water three times, dried with dehydrated magnesium sulfate, and filtered. The resultant product was desolventized using a rotary evaporator. The resultant product was purified using a recrystallization process to obtain 11.3 g of a desired product.

¹H NMR NMR (DMSO, 300 MHz): δ 4.9 (t, 2H), 7.5 (m, 12H), 2.1˜1.76 (m, 18H)

A synthesis process shown in Chemical equation 2c was performed using the compound obtained in Chemical equation 2b.

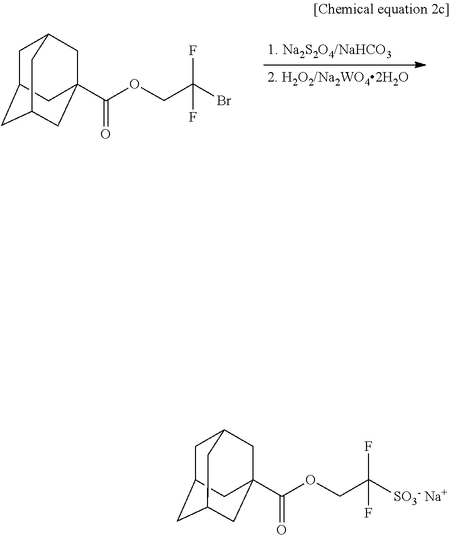

The synthesis process shown in Chemical equation 2c will now be described in further detail. The product obtained in Chemical equation 2b was dissolved in 100 mL of acetonitrile and 100 mL of distilled water in a 500-mL flask. 8.9 g (50 mmol) of sodium hydrosulfite and 6.45 g (76.5 mmol) of sodium bicarbonate were put in the 500-mL flask and stirred while heating the same at a temperature of 60° C. for 20 hours. Subsequently, the resultant product was cooled to ambient temperature, and an organic layer was separated and transferred to a 500-mL flask. Afterwards, 100 mL of distilled water, 4.35 g (3.8 mmol) of 30% hydrogen peroxide, and 40 mg (0.12 mmol) of sodium tungstate dihydrate were put in the 500-mL flask and stirred at ambient temperature for 6 hours. After a reaction was completed, 8.7 g (50 mmol) of sodium hydrosulfite was slowly added to cause a reaction. The remaining hydrogen peroxide was reduced and removed, and a sodium chloride aqueous solution was put in the 500-mL flask to cause layer separation of an organic layer from an aqueous layer. 300 mL of diethyl ether was added to the resultant product and stirred to separate an organic layer, which was at an upper position, from the resultant product. The separated organic layer was dried with dehydrated magnesium sulfate and filtered, and the resultant product was then desolventized using a rotary evaporator. Thereafter, a synthesis process shown in Chemical equation 2d was performed without an additional purification process.

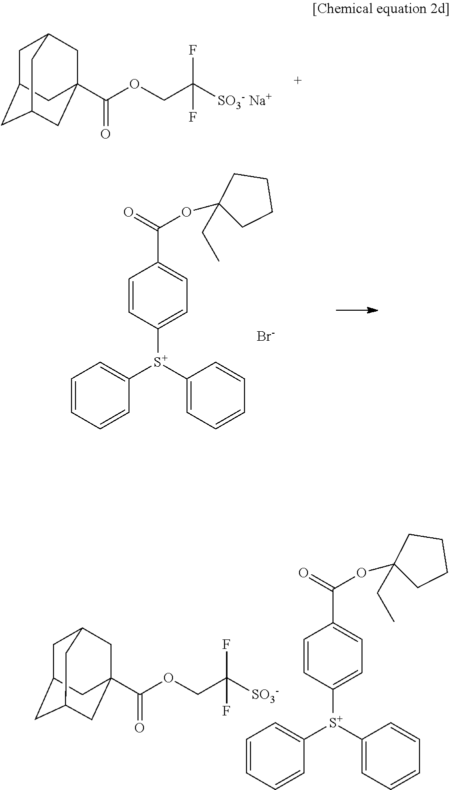

The synthesis process shown in Chemical equation 2d will now be described in detail. 10 g (28.8 mmol) of the product (sodium 2-(((3r,5r,7r)-adamantane-1-carbonyl)oxy)-1,1-difluoroethane-1-sulfonate) obtained in Chemical equation 2c, 50 mL of dichloromethane, and 50 mL of distilled water were put in a 250-mL flask. 14.5 g (30 mmol) of the product (4-(((1-ethylcyclopentyl)oxy)carbonyl)phenyl)diphenylsulfonium bromide) obtained in Chemical equation 2a was added to the 250-mL flask and stirred for 20 hours at ambient temperature. An organic layer (i.e., a dichloromethane layer) at a lower position was separated and washed with 100 mL of distilled water three times. Thereafter, the obtained organic layer was dried with dehydrated magnesium sulfate and filtered. The resultant product was desolventized using a rotary evaporator to be concentrated, and then dissolved again in 20 mL of dichloromethane. 40 mL of diethyl ether was put and cooled to obtain 21.3 g of the compound of Formula 5 ((4-(((1-ethylcyclopentyl)oxy)carbonyl)phenyl)diphenylsulfonium 2-(((3r,5r,7r)-adamantane-1-carbonyl)oxy)-1,1-difluoroethane-1-sulfonate) (yield 95%).

1H NMR (DMSO, 300 MHz): δ 8.0 (d, 2H), 7.44 (m, 12H), 4.96 (t, 2H), 2.1˜1.76 (m, 2H), 2.2˜1.5 (m, 24H), 0.90 (t, 3H)

### Synthesis Example 3

Synthesis of Photo-Decomposable Compound of Formula 6

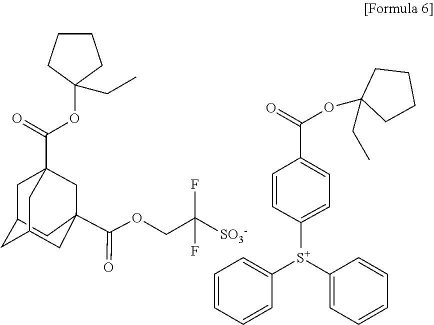

To synthesize the photo-decomposable compound of Formula 6, initially, a first intermediate product (sodium 2-(((1r,3s,5R,7S)-3-(((1-ethylcyclopentyl)oxy)carbonyl)adamantane-1-carbonyl)oxy)-1,1-difluoroethane-1-sulfonate) was synthesized according to the synthesis processes of Chemical equations 1a, 1b, and 1c. Thereafter, a second intermediate product ((4-(((1-ethylcyclopentyl)oxy)carbonyl)phenyl)diphenylsulfonium bromide) was synthesized according to the synthesis process of Chemical equation 2a of Synthesis example 2.

Subsequently, the compound of Formula 6 was synthesized as a final product from the first intermediate product and the second intermediate product according to Chemical equation 3.

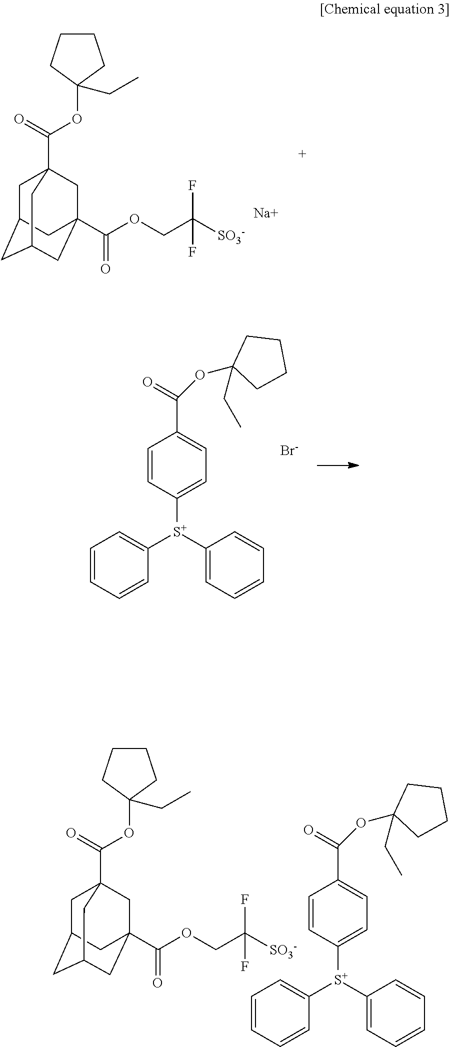

A synthesis process shown in Chemical equation 3 will be described in further detail. 4.2 g (8.6 mmol) of the first intermediate product obtained according to the synthesis process of Chemical equation 1c, 50 mL of dichloromethane, and 50 mL of distilled water were put into a 250-mL flask, 4.8 g (10 mmol) of the second intermediate product obtained according to the synthesis process of Chemical equation 2a was added and stirred at ambient temperature for 20 hours. An organic layer (i.e., a dichloromethane layer) at a lower position was separated, washed with 100 mL of distilled water three times, dried with dehydrated magnesium sulfate, and filtered. The resultant product was desolventized using a rotary evaporator to be concentrated, and then dissolved again in 20 mL of dichloromethane. 40 mL of diethyl ether was put and cooled to obtain 5.2 g of the compound of Formula 6 (yield 70%).

¹H NMR (DMSO, 300 MHz): δ 8.0 (d, 2H), 7.44 (m, 12H), 4.96 (t, 2H), 2.1˜1.76 (m, 2H), 2.2˜1.5 (m, 33H), 0.90 (t, 3H)

By way of summation and review, to help improve the dimensional precision of a pattern required for an IC device in a photolithography process including a positive tone development (PTD) process, a difference in solubility between an exposed area and a non-exposed area of a photoresist film may be increased by increasing a difference in acidity between the exposed area and the non-exposed area of the photoresist film while generating a relatively large amount of acid in the exposed area of the photoresist film with the same amount of light.

One or more embodiments may provide a photo-decomposable compound capable of neutralizing acid.

One or more embodiments may provide a photo-decomposable compound, which may generate acid due to exposure, and increase a difference in solubility in a developer between an exposed area and a non-exposed area of a photoresist film by minimizing a distance by which acid generated due to the exposure diffuses, and a contrast may be improved and the dimensional precision of a pattern required for an integrated circuit (IC) device may be increased.

One or more embodiments may provide a photoresist composition, which may increase a difference in solubility in a developer between an exposed area and a non-exposed area of a photoresist film while generating a relatively large amount of acid in the exposed area of the photoresist film with the same amount of light during a photolithography process, and a contrast may be increased and the dimensional precision of a pattern required for an IC device may be ensured.

One or more embodiments may provide a method of manufacturing an IC device, which may improve the dimensional precision of a pattern during a photolithography process and increase productivity.

Example embodiments have been disclosed herein, and although specific terms are employed, they are used and are to be interpreted in a generic and descriptive sense only and not for purpose of limitation. In some instances, as would be apparent to one of ordinary skill in the art as of the filing of the present application, features, characteristics, and/or elements described in connection with a particular embodiment may be used singly or in combination with features, characteristics, and/or elements described in connection with other embodiments unless otherwise specifically indicated. Accordingly, it will be understood by those of skill in the art that various changes in form and details may be made without departing from the spirit and scope of the present invention as set forth in the following claims.

1. A photo-decomposable compound, comprising:
an anion component including an adamantyl group; and
a cation component including a C5 to C40 cyclic hydrocarbon group, the cation component forming a complex with the anion component,
wherein:
the C5 to C40 cyclic hydrocarbon group has a substituent,
the substituent is configured to decompose in response to exposure to an acid to generate an alkali soluble group, and
the substituent includes an acid-labile protecting group.

2. The photo-decomposable compound as claimed in claim 1,
wherein the acid-labile protecting group is a substituted or unsubstituted t-butyl group or a C3 to C30 substituted or unsubstituted tertiary alicyclic group.

3. The photo-decomposable compound as claimed in claim 1, wherein:
the acid-labile protecting group includes a first substituent,
the first substituent includes a C1 to C10 alkyl group, a C1 to C10 alkoxy group, a halogen atom, a C1 to C10 halogenated alkyl group, a hydroxyl group, an unsubstituted C6 to C30 aryl group, or a C6 to C30 aryl group, and
at least one carbon atom included in the first substituent is substituted with a halogen atom or a heteroatom-containing group.

4. The photo-decomposable compound as claimed in claim 1, wherein:
the substituent has one of the following structures:
*—C(═O)OR¹
*—OC(═O)OR¹
*—OAc,
R¹ is the acid-labile protecting group and includes a substituted or unsubstituted t-butyl group or a C3 to C30 substituted or unsubstituted tertiary alicyclic group,
Ac is an acetal protecting group, and
* indicates a bonding site.

5. The photo-decomposable compound as claimed in claim 4, wherein:
R¹ is a C3 to C30 substituted or unsubstituted tertiary alicyclic group, and
the C3 to C30 substituted or unsubstituted tertiary alicyclic group includes an alicyclic hydrocarbon group, which includes a group in which two hydrogen atoms are excluded from a C3 to C6 monocycloalkane.

6. The photo-decomposable compound as claimed in claim 4, wherein:
R¹ is a C3 to C30 substituted or unsubstituted tertiary alicyclic group, and
the C3 to C30 substituted or unsubstituted tertiary alicyclic group includes an alicyclic hydrocarbon group, which includes a group in which two hydrogen atoms are excluded from a C7 to C12 polycycloalkane.

7. The photo-decomposable compound as claimed in claim 1, wherein:
the photo-decomposable compound is represented by Formula 2: 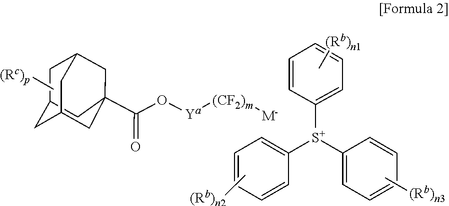
in Formula 2,
R^{b} is the substituent, and is represented by *—C(═O)OR¹, in which R¹ is the acid-labile protecting group and includes a substituted or unsubstituted t-butyl or a C3 to C30 substituted or unsubstituted tertiary alicyclic group,
Y^{a} is a C1 to C20 divalent linear or cyclic hydrocarbon group,
m is an integer of 1 to 5,
n1, n2, and n3 are each independently an integer of 0 to 2, at least one of n1, n2, and n3 being 1 or 2,
R^{c} is a C1 to C10 alkyl group, a C1 to C10 alkoxy group, a halogen atom, a C1 to C10 halogenated alkyl group, a hydroxyl group, an unsubstituted C6 to C30 aryl group, or a C6 to C30 aryl group in which at least one carbon atom included in R^{c} is substituted with a halogen atom or a heteroatom-containing group,
p is an integer of 0 to 2, and
M⁻ is —SO₃⁻ or —CO₂⁻.

8. The photo-decomposable compound as claimed in claim 7, wherein R¹ is one of the following structures: 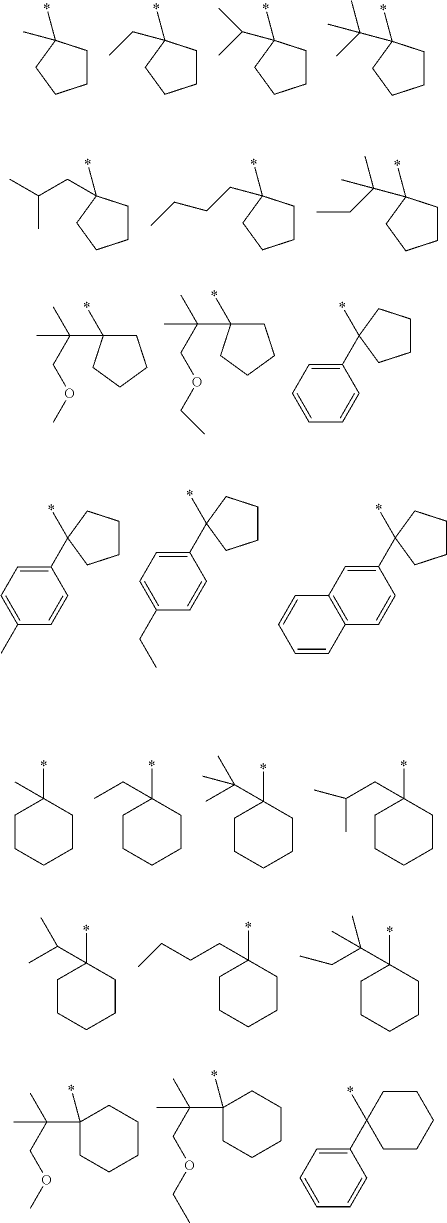 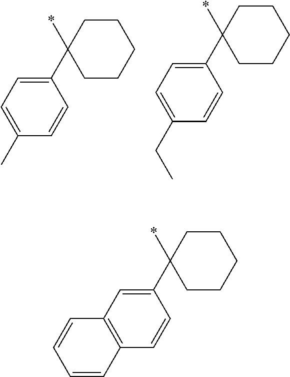 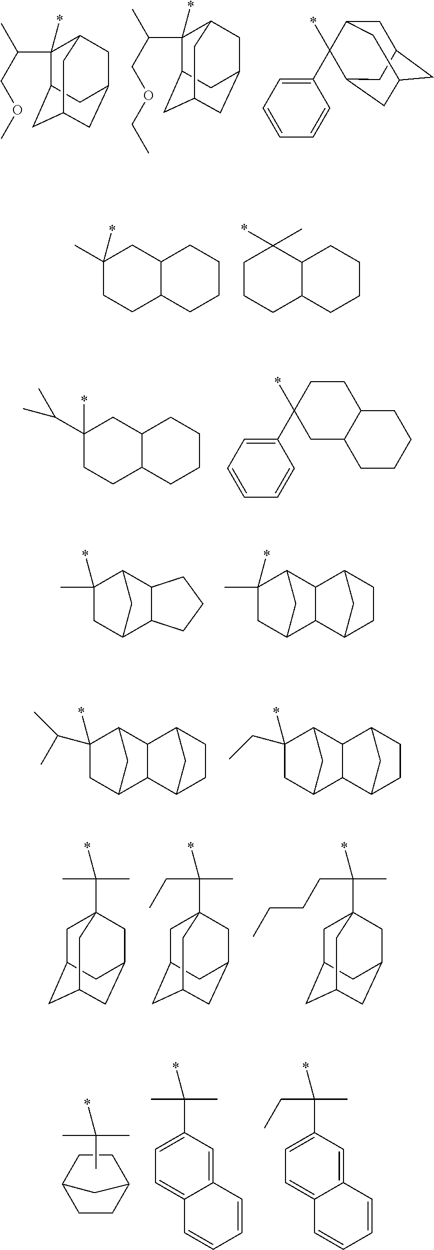 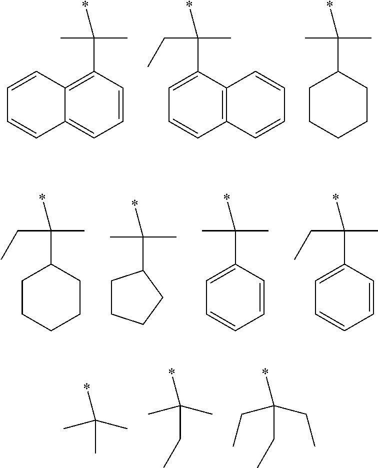
in which * indicates a bonding site.

9. The photo-decomposable compound as claimed in claim 7, wherein R¹ is one of the following structures: 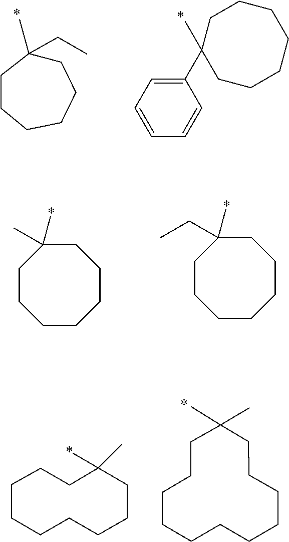
in which * indicates a bonding site.

10. The photo-decomposable compound as claimed in claim 7, wherein R¹ is one of the following structures: 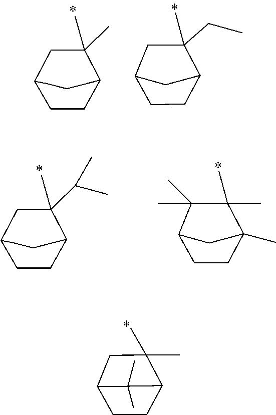
in which * indicates a bonding site.

11. The photo-decomposable compound as claimed in claim 7, wherein R¹ is one of the following structures: 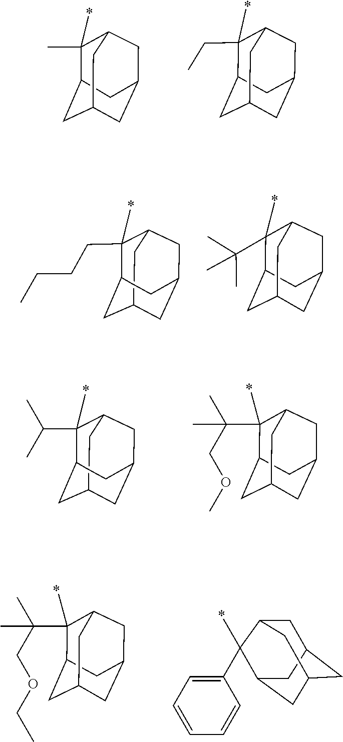
in which * indicates a bonding site.

12. The photo-decomposable compound as claimed in claim 7, wherein R¹ is one of the following structures: 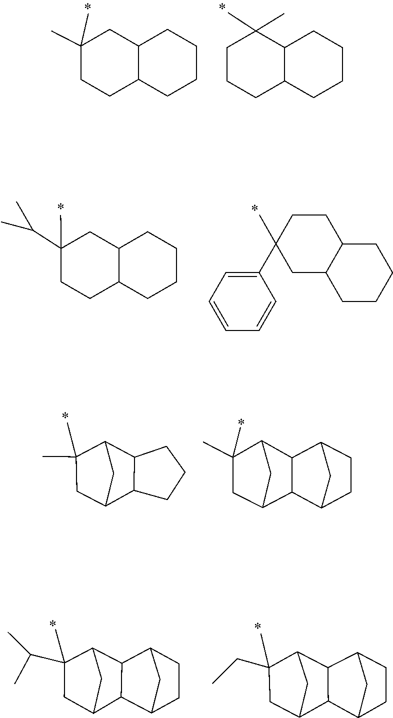
in which * indicates a bonding site.

13. The photo-decomposable compound as claimed in claim 7, wherein R¹ is one of the following structures: 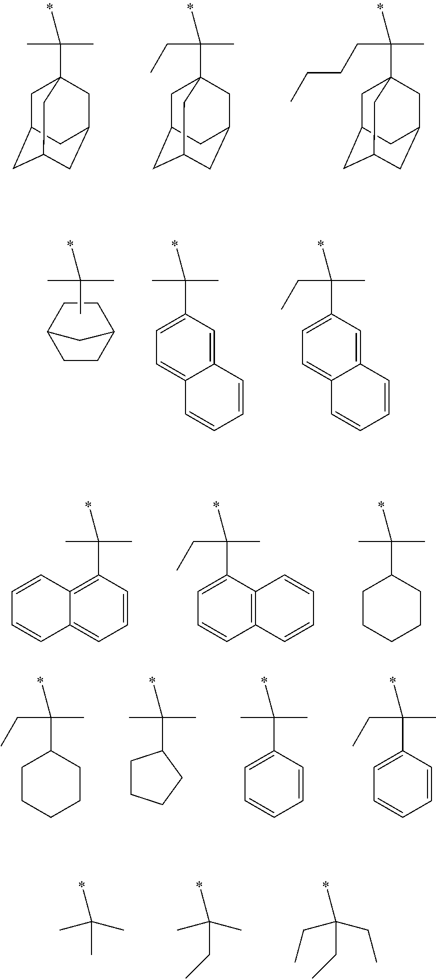
wherein * indicates a bonding site.

14. The photo-decomposable compound as claimed in claim 7, wherein Y^{a} is a C1 to C5 substituted or unsubstituted alkylene group, a C5 to C20 divalent monocyclic or condensed alicyclic hydrocarbon group, or a C5 to C20 divalent monocyclic or condensed aromatic hydrocarbon group.

15. The photo-decomposable compound as claimed in claim 7, wherein Y^{a} is —(CH₂)ₙ—, in which n is an integer of 1 to 5.

16. The photo-decomposable compound as claimed in claim 7, wherein Y^{a} is —(CH₂)ₙ—, in which n is an integer of 1 to 5.

16. The photo-decomposable compound as claimed in claim 7, wherein:
Y^{a} is one of the following structures: 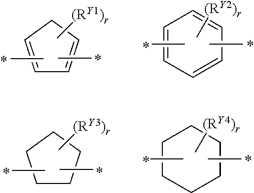
* indicates a bonding site,
r is an integer of 0 to 2, and
R^{Y1}, R^{Y2}, R^{Y3}, and R^{Y4} are each independently a C1 to C10 linear or branched alkyl group, a cyclopropyl group, a cyclopentyl group, or a cyclohexyl group.

17. The photo-decomposable compound as claimed in claim 1, wherein:
the photo-decomposable compound is represented by Formula 3: 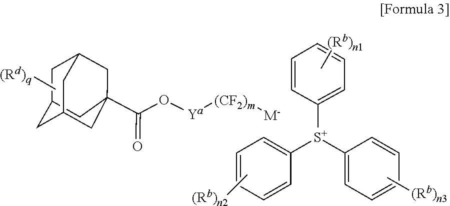
in Formula 3,
each of R^{b} and R^{d} is the substituent, and is represented by *—C(═O)OR¹, in which R¹ is the acid-labile protecting group and includes a substituted or unsubstituted t-butyl or a C3 to C30 substituted or unsubstituted tertiary alicyclic group,
Y^{a} is a C1 to C20 divalent linear or cyclic hydrocarbon group,
m is an integer of 1 to 5,
n1, n2, and n3 are each independently an integer of 0 to 2, at least one of n1, n2, and n3 being 1 or 2,
q is 1 or 2, and
M⁻ is —SO₃⁻ or —CO₂⁻.

18. The photo-decomposable compound as claimed in claim 17, wherein R¹ is one of the following structures: 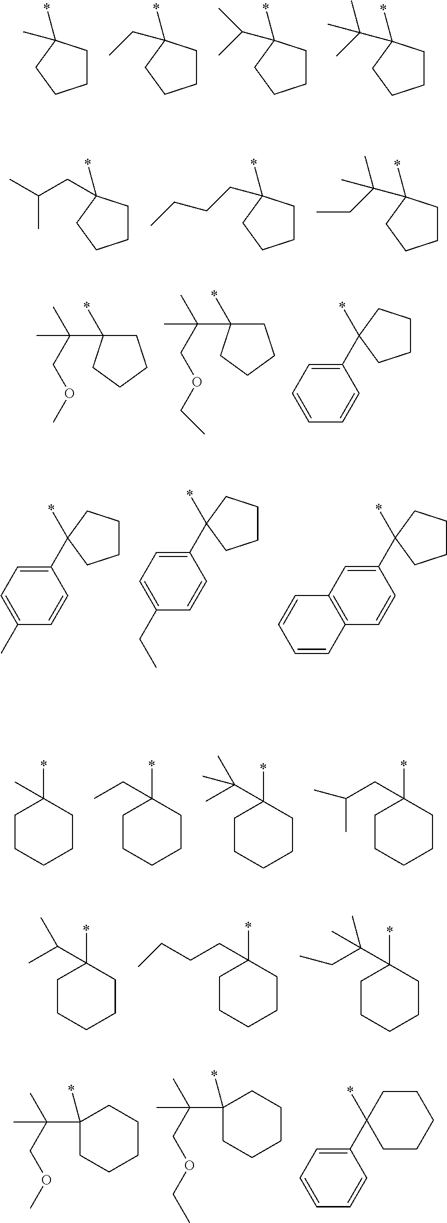 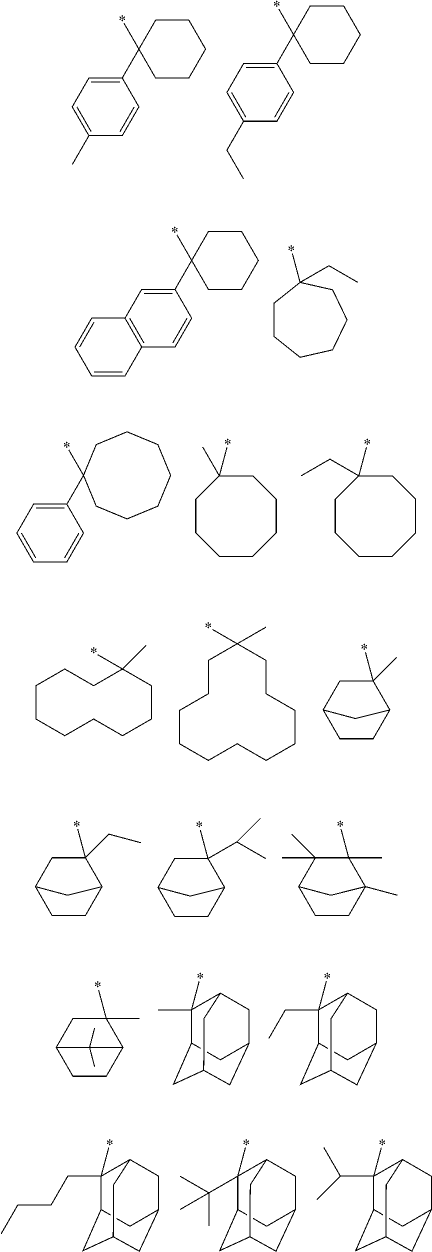 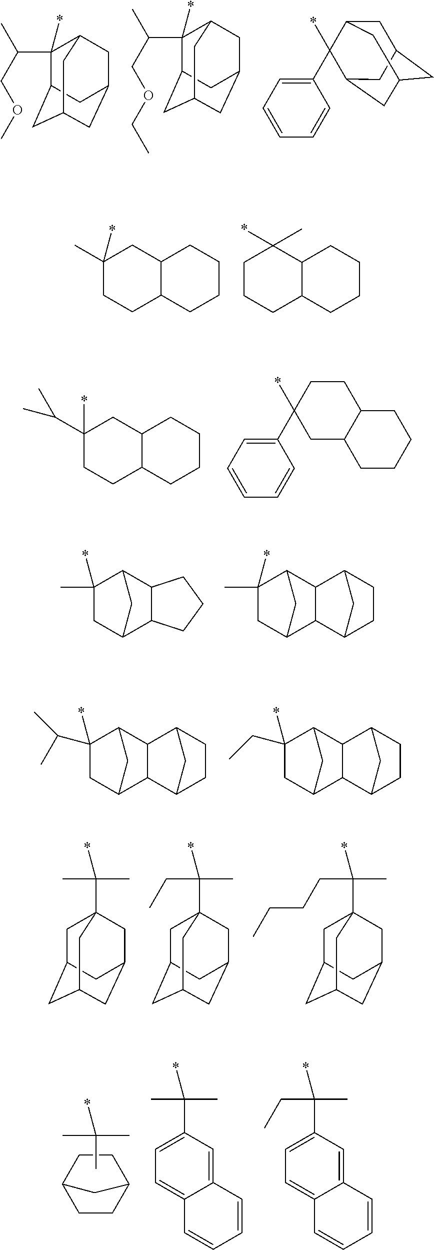 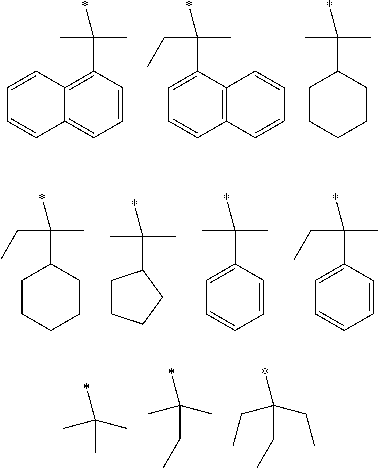
wherein * indicates a bonding site.

19. The photo-decomposable compound as claimed in claim 17, wherein:
Y^{a} is a C1 to C5 substituted or unsubstituted alkylene group, a C5 to C20 divalent monocyclic or condensed alicyclic hydrocarbon group, a C5 to C20 divalent monocyclic or condensed aromatic hydrocarbon group, —(CH₂)ₙ—, in which n is an integer of 1 to 5, or one of the following structures: 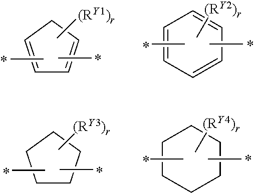
* indicates a bonding site,
r is an integer of 0 to 2, and
R^{Y1}, R^{Y2}, R^{Y3}, and R^{Y4} are each independently a C1 to C10 linear or branched alkyl group, a cyclopropyl group, a cyclopentyl group, or a cyclohexyl group.

20. The photo-decomposable compound as claimed in claim 1, wherein the photo-decomposable compound is represented by Formula 5 or Formula 6:

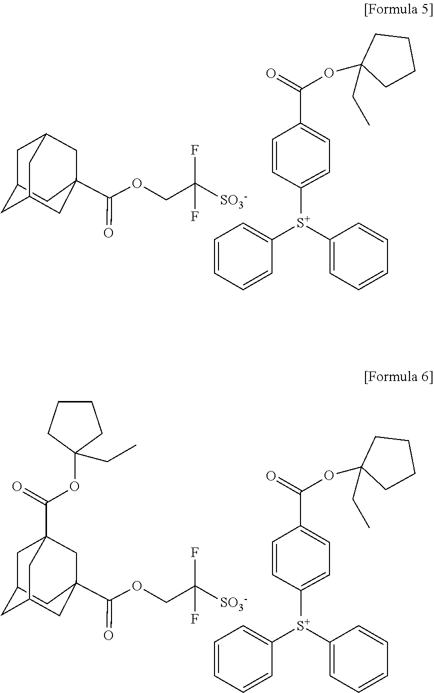

21.-40. (canceled)
